(19) United States
(12) Patent Application Publication
(10) Pub. No.: US 2009/0197878 A1
(43) Pub. Date: Aug. 6, 2009

(54) SUBSTITUTED BENZO[d][1,3]OXAZIN-2(4H)-ONES AND RELATED DERIVATIVES AND THEIR USES FOR MODULATING THE PROGESTERONE RECEPTOR

(75) Inventors: Thomas Joseph Commons, Wayne, PA (US); Douglas John Jenkins, Cincinatti, OH (US); Eugene John Trybulski, Huntingdon Valley, PA (US); Andrew Fensome, Wayne, PA (US)

Correspondence Address: HOWSON & HOWSON LLP / WYETH, 501 OFFICE CENTER DRIVE, SUITE 210, FORT WASHINGTON, PA, 19034, US

(73) Assignee: Wyeth, Madison, NJ (US)

(21) Appl. No.: 12/361,764

(22) Filed: Jan. 29, 2009

Related U.S. Application Data
(60) Provisional application No. 61/025,368, filed on Feb. 1, 2008.

(51) Int. Cl. A61K 31/536; C07D 215/227; C07D 265/18; A61P 15/00; A61K 31/537; A61K 31/4704

(52) U.S. Cl. 514/230.5; 546/158; 544/92; 544/71; 514/312

(57) ABSTRACT

Compounds of formula (I), or pharmaceutically acceptable salts thereof, are provided, wherein R₁-R₆ and X are defined herein. Also provided are methods of preparing the compounds of formula (I), pharmaceutical compositions and kits containing a compound of formula (I), as are methods of treating endometriosis, hormone-dependent carcinomas, leiomyoma, fibroids, dysfunctional bleeding, polycystic ovary syndrome, and menopause related symptoms; methods of contraception; methods of providing hormone replacement therapy; methods of stimulating food intake; methods of synchronizing estrus; and methods of treating symptoms of premenstrual syndrome and premenstrual dysphoric disorder by administering to a mammal in need thereof a pharmaceutically effective amount of a compound of formula (I).

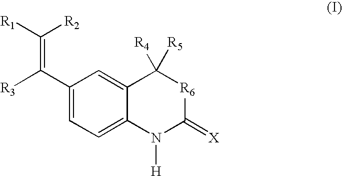

### CROSS-REFERENCE TO RELATED APPLICATION

This application claims the benefit of the priority of U.S. Provisional Patent Application No. 61/025,368, filed Feb. 1, 2008.

### BACKGROUND OF THE INVENTION

This invention relates to modulators of the progesterone receptor, their preparation and utility.

Intracellular receptors (IR) form a class of structurally related gene regulators known as “ligand dependent transcription factors” (Mangelsdorf, D. J. etc. Cell, 83, 835, 1995). The steroid receptor family is a subset of the IR family, including the progesterone receptor (PR), estrogen receptor (ER), androgen receptor (AR), glucocorticoid receptor (GR), and mineralocorticoid receptor (MR).

The natural hormone, or ligand, for the PR is the steroid progesterone, but synthetic compounds, such as medroxyprogesterone acetate or levonorgestrel, have been made which also serve as PR ligands. Once a ligand is present in the fluid surrounding a cell, it passes through the membrane via passive diffusion, and binds to the IR to create a receptor/ligand complex. This complex binds to specific gene promoters present in the cell's DNA. Once bound to the DNA, the complex modulates the production of mRNA and the protein encoded by that gene.

PR agonists (progestins) and PR antagonists (antiprogestins) are known in the art and include those described in U.S. Pat. Nos. 6,509,334; 6,436,929; 6,380,235; 6,358,948; and 6,407,101 and US Patent Application Publication No. US-2006/0035843.

What is needed in the art are alternative PR modulators.

### SUMMARY OF THE INVENTION

In one aspect, compounds of formula (I) are provided, wherein R₁-R₆ and X are defined herein, wherein R₁ is selected from among H, 13 CO₂—C₁—C₃ alkyl, —CO₂H, —CR₇O, —CONR₈R₉, —CN, and isoxazole; R₂ is selected from among H, —CO₂—C₁—C₃-alkyl, —CO₂H, —CR₇O, —CONR₈R₉, —CN, C₁-C₆ alkyl, aryl, and heteroaryl; with the proviso that both R₁ and R₂ are not H; R₃ is H, C₁-C₆ alkyl, aryl, or heteroaryl; or R₁ and R₃ together form a 6-membered lactone, wherein a 2-carbon bridge is formed between the CO₂—C₁—C₃ alkyl of R₁ and an alkyl group of R₃; and R₄-R₆ and X are defined below.

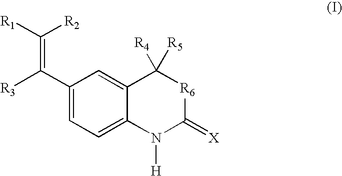

In another aspect, compounds of formula (Ia) are provided, wherein R₁ is selected from among H, —CO₂—C₁—C₃ alkyl, —CO₂H, —CR₇O, —CONR₈R₉, —CN, and isoxazole; R₂ is selected from among H, —CO₂—C₁—C₃-alkyl, —CO₂H, —CR₇O, —CONR₈R₉, —CN, C₁-C₆ alkyl, aryl, and heteroaryl; with the proviso that both R₁ and R₂ are not H; R₃ is H, C₁-C₆ alkyl, aryl, or heteroaryl; and R₄-R₆ and X are defined herein.

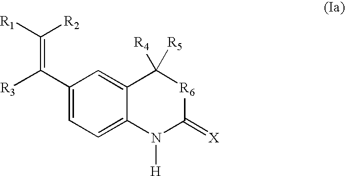

In a further aspect, compounds of formula (Ib) are provided, wherein R₁ and R₃ together form a 6-membered lactone, wherein a 2-carbon bridge is formed between the CO₂—C₁—C₃ alkyl of R₁ and an alkyl group of R₃; R₂ is selected from among H, —CO₂—C₁—C₃-alkyl, —CO₂H, —CR₇O, —CONR₈R₉, —CN, C₁-C₆ alkyl, aryl, and heteroaryl; and R₄-R₆ and X are defined herein.

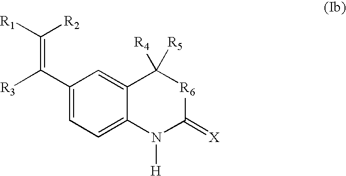

In another aspect, methods for preparing compounds of formula (I), (Ia), and (Ib) are provided.

In a further aspect, a pharmaceutical composition is provided and contains a compound of formula (I), (Ia), and/or (Ib).

In still another embodiment, methods of treating endometriosis, hormone-dependent carcinomas, leiomyoma, fibroids, dysfunctional bleeding, polycystic ovary syndrome, and menopause related symptoms; methods of contraception; methods of providing hormone replacement therapy; methods of stimulating food intake; methods of synchronizing estrus; and methods of treating symptoms of premenstrual syndrome and premenstrual dysphoric disorder are provided and include administering to a mammal in need thereof a pharmaceutically effective amount of a compound of formula (I), (Ia), and/or (Ib).

In yet a further aspect, kits containing a compound of formula (I), (Ia), and/or (Ib) and a carrier suitable for administration to a mammalian subject are provided.

Other aspects and advantages of the invention will be readily apparent from the following detailed description of the invention.

### DETAILED DESCRIPTION OF THE INVENTION

Compounds of formula (I), or a pharmaceutically acceptable salt thereof, are provided.

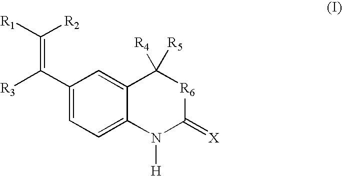

R₁ is selected from among H, —CO₂—C₁—C₃ alkyl, —CO₂H, —CR₇O, —CONR₈R₉, —CN, and isoxazole; R₂ is selected from among H, —CO₂—C₁—C₃-alkyl, —CO₂H, —CR₇O, —CONR₈R₉, —CN, C₁-C₆ alkyl, aryl, and heteroaryl; with the proviso that both R₁ and R₂ are not H; R₃ is H, C₁-C₆ alkyl, aryl, or heteroaryl; or R₁ and R₃ together form a 6-membered lactone, wherein a 2-carbon bridge is formed between the CO₂—C₁—C₃ alkyl of R₁ and an alkyl group of R₃; R4 and R₅ are independently C₁-C₃ alkyl or together with the carbon to which they are attached form a saturated 3 to 6-membered carbon-based ring; R₆ is O or CR₁₀R₁₁; R₇, R₈ and R₉ are independently selected from among H and C₁-C₃ alkyl; R₁₀ and R₁₁ are independently H or C₁-C₃ alkyl; or R₁₀ and R₁₁ together with the carbon to which they are attached form a saturated 3 to 6-membered carbon-based ring; and X is O or S.

In one embodiment, compounds of formula (Ia), or a pharmaceutically acceptable salt thereof, are provided.

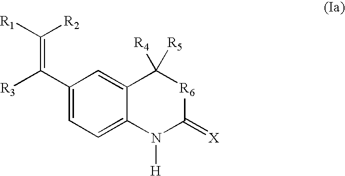

wherein, R₁ is selected from among H, —CO₂—C₁—C₃ alkyl, —CO₂H, —CR₇O, —CONR₈R₉, —CN, and isoxazole; R₂ is selected from among H, —CO₂—C₁—C₃-alkyl, —CO₂H, —CR₇O, —CONR₈R₉, —CN, C₁-C₆ alkyl, aryl, and heteroaryl; with the proviso that both R₁ and R₂ are not H; R₃ is H, C₁-C₆ alkyl, aryl, or heteroaryl; R₄ and R₅ are independently C₁-C₃ alkyl or together with the carbon to which they are attached form a saturated 3 to 6-membered carbon-based ring; R₆ is O or CR₁₀R₁₁; R₇, R₈ and R₉ are independently selected from among H and C₁-C₃ alkyl; R₁₀ and R₁₁ are independently H or C₁-C₃ alkyl; or R₁₀ and R₁₁ together with the carbon to which they are attached form a saturated 3 to 6-membered carbon-based ring; and X is O or S.

In a further embodiment, compounds of formula (Ib), or a pharmaceutically acceptable salt thereof, are provided.

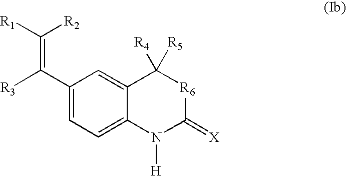

wherein, R₁ and R₃ together form a 6-membered lactone, wherein a 2-carbon bridge is formed between the CO₂—C₁—C₃ alkyl of R₁ and an alkyl group of R₃; R₂ is selected from among H, —CO₂—C₁—C₃-alkyl, —CO₂H, —CR₇O, —CONR₈R₉, —CN, C₁-C₆ alkyl, aryl, and heteroaryl; R₄ and R₅ are independently C₁-C₃ alkyl or together with the carbon to which they are attached form a saturated 3 to 6-membered carbon-based ring; R₆ is O or CR₁₀R₁₁; R₇, R₈ and R₉ are independently selected from among H and C₁-C₃ alkyl; R₁₀ and R₁₁ are independently H or C₁-C₃ alkyl; or R₁₀ and R₁₁ together with the carbon to which they are attached form a saturated 3 to 6-membered carbon-based ring; and X is O or S.

In a further embodiment, R₁ is CN.

In another embodiment, R₄ and R₅ are methyl.

In a further embodiment, R₄ and R₅ are ethyl.

In a yet further embodiment, R₂ is aryl or substituted aryl. Desirably, the R₂ aryl is phenyl, substituted phenyl, naphthyl or substituted naphthyl. More desirably, the R₂ aryl is phenyl, phenyl substituted with C₁-C₆ alkyl, naphthyl or naphthyl substituted with C₁-C₆ alkyl.

In still another embodiment, R₂ is heteroaryl or substituted heteroaryl. Desirably, the R₂ heteroaryl is pyridine, substituted pyridine, thiophene, substituted thiophene, furan, substituted furan, pyrrole, or substituted pyrrole.

In still another embodiment, R₂ is C₁ to C₆ alkyl. Desirably R₂ is C₁ to C₃ alkyl.

In still another embodiment, R₃ is C₁ to C₆ alkyl. Desirably R₃ is C₁ to C₃ alkyl.

In still a further embodiment, R₃ is aryl or substituted aryl. Desirably, the R₃ aryl is phenyl, substituted phenyl, naphthyl or substituted naphthyl. More desirably, the R₃ aryl is phenyl, phenyl substituted with C₁-C₆ alkyl, naphthyl or naphthyl substituted with C₁-C₆ alkyl.

In yet another embodiment, R₃ is heteroaryl or substituted heteroaryl. Desirably, the R₃ heteroaryl is pyridine, substituted pyridine, thiophene, substituted thiophene, furan, substituted furan, pyrrole, or substituted pyrrole.

In another embodiment, X is O.

In still a further embodiment, R₁ and R₃ are joined to form a 6-membered lactone. Desirably, the 6-membered lactone is of the structure:

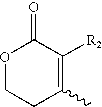

The compound of formula (I) may therefore be selected from among Methyl-3-(4,4-dimethyl-2-oxo-1,2,3,4-tetrahydroquinolin-6-yl)acrylate; Methyl-3-(4,4-dimethyl-2-oxo-1,4-dihydro-2H-3,1-benzoxazin-6-yl)acrylate; 6-[2-Isoxazol-5-ylvinyl]-4,4-dimethyl-1,4-dihydro-2H-3,1-benzoxazin-2-one; 3-(4,4-Dimethyl-2-oxo-1,4-dihydro-2H-3,1-benzoxazin-6-yl)acrylamide; 3-(4,4-Dimethyl-2-oxo-1,4-dihydro-2H-3,1-benzoxazin-6-yl)but-2-enamide; 3-(4,4-Dimethyl-2-oxo-1,4-dihydro-2H-3,1-benzoxazin-6-yl)-3-phenylacrylonitrile; 3-(4,4-Dimethyl-2-oxo-1,4-dihydro-2H-3,1-benzoxazin-6-yl)-3-phenylacrylamide; 3-(4,4-Dimethyl-2-oxo-1,4-dihydro-2H-3,1-benzoxazin-6-yl)acrylonitrile; Methyl-3-(4,4-dimethyl-2-oxo-1,4-dihydro-2H-3,1-benzoxazin-6-yl)but-2-enoate; 4,4-Dimethyl-6-(6-oxo-3,6-dihydro-2H-pyran-4-yl)-1,4-dihydro-2H-3,1-benzoxazin-2-one; Methyl-3-(4,4-dimethyl-2-oxo-1,4-dihydro-2H-3,1-benzoxazin-6-yl)-3-phenylacrylate; 3-(4,4-Dimethyl-2-oxo-1,4-dihydro-2H-3,1-benzoxazin-6-yl)but-2-enenitrile; 3-(4,4-Dimethyl-2-oxo-1,4-dihydro-2H-3,1-benzoxazin-6-yl)acrylic acid; 3-(4,4-Dimethyl-2-oxo-1,4-dihydro-2H-3,1-benzoxazin-6-yl)but-2-enoic acid; 3-(2-Oxo-1,2-dihydrospiro[3,1-benzoxazine-4,1′-cyclohexan]-6-yl)but-2-enamide; 3-(2-oxo-1,2-dihydrospiro[3,1-benzoxazine-4,1′-cyclohexan]-6-yl)but-2-enenitrile; 3-(2-Oxo-1,2-dihydrospiro[3,1-benzoxazine-4,1′-cyclohexan]-6-yl)pent-2-enamide; 3-(2-Oxo-1,2-dihydrospiro[3,1-benzoxazine-4,1′-cyclohexan]-6-yl)pent-2-enenitrile; 3-(2-Oxo-1,2-dihydrospiro[3,1-benzoxazine-4,1′-cyclohexan]-6-yl)hex-2-enenitrile; 4-methyl-3-(2-oxo-1,2-dihydrospiro[3,1-benzoxazine-4,1′-cyclohexan]-6-yl)pent-2-enenitrile; 3-(4,4-Diethyl-2-oxo-1,4-dihydro-2H-3,1-benzoxazin-6-yl)but-2-enenitrile; 3-(4,4-Diethyl-2-oxo-1,4-dihydro-2H-3,1-benzoxazin-6-yl)pent-2-enenitrile; 3-(4,4-Diethyl-2-oxo-1,4-dihydro-2H-3,1-benzoxazin-6-yl)hex-2-enamide; 3-(4,4-Diethyl-2-oxo-1,4-dihydro-2H-3,1-benzoxazin-6-yl)hex-2-enenitrile; 3-(4,4-Diethyl-2-oxo-1,4-dihydro-2H-3,1-benzoxazin-6-yl)hex-2-enal; 3-(4,4-Diethyl-2-oxo-1,4-dihydro-2H-3,1-benzoxazin-6-yl)-4-methylpent-2-enenitrile; 3-(4,4-Dimethyl-2-oxo-1,4-dihydro-2H-3,1-benzoxazin-6-yl)pent-2-enenitrile; (2E)-3-(4,4-Dimethyl-2-oxo-1,4-dihydro-2H-3,1-benzoxazin-6-yl)hex-2-enenitrile; and 3-(4,4-Dimethyl-2-oxo-1,4-dihydro-2H-3,1-benzoxazin-6-yl)-4-methylpent-2-enenitrile, and pharmaceutically acceptable salts thereof.

The compounds as described can contain one or more asymmetric centers and can thus give rise to optical isomers and diastereomers. The compounds can include optical isomers and diastereomers; racemic and resolved enantiomerically pure R and S stereoisomers; other mixtures of the R and S stereoisomers; mixtures of E and Z isomers; and pharmaceutically acceptable salts thereof. In one embodiment, the compounds have one or more carbon-carbon double bonds with E stereochemistry. In another embodiment, the compounds have one or more carbon-carbon double bonds with Z stereochemistry. In a further embodiment, R₁ has E stereochemistry relative to the aryl ring containing the R₄R₅ substituents. In still another embodiment, R₁ has Z stereochemistry relative to the aryl ring containing the R₄R₅ substituents.

Desirably, the compound of formula (I) is selected from among Methyl(2E)-3-(4,4-dimethyl-2-oxo-1,2,3,4-tetrahydroquinolin-6-yl)acrylate; Methyl(2E)-3-(4,4-dimethyl-2-oxo-1,4-dihydro-2H-3,1-benzoxazin-6-yl)acrylate; 6-[(E)-2-Isoxazol-5-ylvinyl]-4,4-dimethyl-1,4-dihydro-2H-3,1-benzoxazin-2-one; (2E)-3-(4,4-Dimethyl-2-oxo-1,4-dihydro-2H-3,1-benzoxazin-6-yl)acrylamide; (2E)-3-(4,4-Dimethyl-2-oxo-1,4-dihydro-2H-3,1-benzoxazin-6-yl)but-2-enamide; (2E)-3-(4,4-Dimethyl-2-oxo-1,4-dihydro-2H-3,1-benzoxazin-6-yl)-3-phenylacrylonitrile; (2E)-3-(4,4-Dimethyl-2-oxo-1,4-dihydro-2H-3,1-benzoxazin-6-yl)-3-phenylacrylamide; (2E)-3-(4,4-Dimethyl-2-oxo-1,4-dihydro-2H-3,1-benzoxazin-6-yl)acrylonitrile; Methyl(2E)-3-(4,4-dimethyl-2-oxo-1,4-dihydro-2H-3,1-benzoxazin-6-yl)but-2-enoate; Methyl(2E)-3-(4,4-dimethyl-2-oxo-1,4-dihydro-2H-3,1-benzoxazin-6-yl)-3-phenylacrylate; (2E)-3-(4,4-Dimethyl-2-oxo-1,4-dihydro-2H-3,1-benzoxazin-6-yl)but-2-enenitrile; (2E)-3-(4,4-Dimethyl-2-oxo-1,4-dihydro-2H-3,1-benzoxazin-6-yl)acrylic acid; (2E)-3-(4,4-Dimethyl-2-oxo-1,4-dihydro-2H-3,1-benzoxazin-6-yl)but-2-enoic acid; (2E)-3-(2-Oxo-1,2-dihydrospiro[3,1-benzoxazine-4,1′-cyclohexan]-6-yl)but-2-enamide; (2E)-3-(2-oxo-1,2-dihydrospiro[3,1-benzoxazine-4,1′-cyclohexan]-6-yl)but-2-enenitrile; (2E)-3-(2-Oxo-1,2-dihydrospiro[3,1-benzoxazine-4,1′-cyclohexan]-6-yl)pent-2-enamide; (2E)-3-(2-Oxo-1,2-dihydrospiro[3,1-benzoxazine-4,1′-cyclohexan]-6-yl)pent-2-enenitrile; (2E)-3-(2-Oxo-1,2-dihydrospiro[3,1-benzoxazine-4,1′-cyclohexan]-6-yl)hex-2-enenitrile; (2E)-4-methyl-3-(2-oxo-1,2-dihydrospiro[3,1-benzoxazine-4,1′-cyclohexan]-6-yl)pent-2-enenitrile; (2E)-3-(4,4-Diethyl-2-oxo-1,4-dihydro-2H-3,1-benzoxazin-6-yl)but-2-enenitrile; (2E)-3-(4,4-Diethyl-2-oxo-1,4-dihydro-2H-3,1-benzoxazin-6-yl)pent-2-enenitrile; (2E)-3-(4,4-Diethyl-2-oxo-1,4-dihydro-2H-3,1-benzoxazin-6-yl)hex-2-enamide; (2E)-3-(4,4-Diethyl-2-oxo-1,4-dihydro-2H-3,1-benzoxazin-6-yl)hex-2-enenitrile; (2E)-3-(4,4-Diethyl-2-oxo-1,4-dihydro-2H-3,1-benzoxazin-6-yl)hex-2-enal; (2E)-3-(4,4-Diethyl-2-oxo-1,4-dihydro-2H-3,1-benzoxazin-6-yl)-4-methylpent-2-enenitrile; (2E)-3-(4,4-Dimethyl-2-oxo-1,4-dihydro-2H-3,1-benzoxazin-6-yl)pent-2-enenitrile; (2E)-3-(4,4-Dimethyl-2-oxo-1,4-dihydro-2H-3,1-benzoxazin-6-yl)hex-2-enenitrile; (2E)-3-(4,4-Dimethyl-2-oxo-1,4-dihydro-2H-3,1-benzoxazin-6-yl)-4-methylpent-2-enenitrile, and pharmaceutically acceptable salts thereof.

The compound of formula (I) may further be selected from among Methyl (2Z)-3-(4,4-dimethyl-2-oxo-1,2,3,4-tetrahydroquinolin-6-yl)acrylate; Methyl(2Z)-3-(4,4-dimethyl-2-oxo-1,4-dihydro-2H-3,1-benzoxazin-6-yl)acrylate; 6-[(Z)-2-Isoxazol-5-ylvinyl]-4,4-dimethyl-1,4-dihydro-2H-3,1-benzoxazin-2-one; (2Z)-3-(4,4-Dimethyl-2-oxo-1,4-dihydro-2H-3,1-benzoxazin-6-yl)acrylamide; (2Z)-3-(4,4-Dimethyl-2-oxo-1,4-dihydro-2H-3,1-benzoxazin-6-yl)but-2-enamide; (2Z)-3-(4,4-Dimethyl-2-oxo-1,4-dihydro-2H-3,1-benzoxazin-6-yl)-3-phenylacrylonitrile; (2Z)-3-(4,4-Dimethyl-2-oxo-1,4-dihydro-2H-3,1-benzoxazin-6-yl)-3-phenylacrylamide; (2Z)-3-(4,4-Dimethyl-2-oxo-1,4-dihydro-2H-3,1-benzoxazin-6-yl)acrylonitrile; Methyl(2Z)-3-(4,4-dimethyl-2-oxo-1,4-dihydro-2H-3,1-benzoxazin-6-yl)but-2-enoate; Methyl(2Z)-3-(4,4-dimethyl-2-oxo-1,4-dihydro-2H-3,1-benzoxazin-6-yl)-3-phenylacrylate; (2Z)-3-(4,4-Dimethyl-2-oxo-1,4-dihydro-2H-3,1-benzoxazin-6-yl)but-2-enenitrile; (2Z)-3-(4,4-Dimethyl-2-oxo-1,4-dihydro-2H-3,1-benzoxazin-6-yl)acrylic acid; (2Z)-3-(4,4-Dimethyl-2-oxo-1,4-dihydro-2H-3,1-benzoxazin-6-yl)but-2-enoic acid; (2Z)-3-(2-Oxo-1,2-dihydrospiro[3,1-benzoxazine-4,1′-cyclohexan]-6-yl)but-2-enamide; (2Z)-3-(2-oxo-1,2-dihydrospiro[3,1-benzoxazine-4,1′-cyclohexan]-6-yl)but-2-enenitrile; (2Z)-3-(2-Oxo-1,2-dihydrospiro[3,1-benzoxazine-4,1′-cyclohexan]-6-yl)pent-2-enamide; (2Z)-3-(2-Oxo-1,2-dihydrospiro[3,1-benzoxazine-4,1′-cyclohexan]-6-yl)pent-2-enenitrile; (2Z)-3-(2-Oxo-1,2-dihydrospiro[3,1-benzoxazine-4,1′-cyclohexan]-6-yl)hex-2-enenitrile; (2Z)-4-methyl-3-(2-oxo-1,2-dihydrospiro[3,1-benzoxazine-4,1′-cyclohexan]-6-yl)pent-2-enenitrile; (2Z)-3-(4,4-Diethyl-2-oxo-1,4-dihydro-2H-3,1-benzoxazin-6-yl)but-2-enenitrile; (2Z)-3-(4,4-Diethyl-2-oxo-1,4-dihydro-2H-3,1-benzoxazin-6-yl)pent-2-enenitrile; (2Z)-3-(4,4-Diethyl-2-oxo-1,4-dihydro-2H-3,1-benzoxazin-6-yl)hex-2-enamide; (2Z)-3-(4,4-Diethyl-2-oxo-1,4-dihydro-2H-3,1-benzoxazin-6-yl)hex-2-enenitrile; (2Z)-3-(4,4-Diethyl-2-oxo-1,4-dihydro-2H-3,1-benzoxazin-6-yl)hex-2-enal; (2Z)-3-(4,4-Diethyl-2-oxo-1,4-dihydro-2H-3,1-benzoxazin-6-yl)-4-methylpent-2-enenitrile; (2Z)-3-(4,4-Dimethyl-2-oxo-1,4-dihydro-2H-3,1-benzoxazin-6-yl)pent-2-enenitrile; (2Z)-3-(4,4-Dimethyl-2-oxo-1,4-dihydro-2H-3,1-benzoxazin-6-yl)hex-2-enenitrile; (2Z)-3-(4,4-Dimethyl-2-oxo-1,4-dihydro-2H-3,1-benzoxazin-6-yl)-4-methylpent-2-enenitrile, and pharmaceutically acceptable salts thereof.

The term “alkyl” is used herein to refer to both straight- and branched-chain saturated aliphatic hydrocarbon groups. In one embodiment, an alkyl group has 1 to 8 carbon atoms (i.e., C₁, C₂, C₃, C₄, C₅ C₆, C₇, or C₈). In another embodiment, an alkyl group has 1 to 6 carbon atoms (i.e., C₁, C₂, C₃, C₄, C₅ or C₆). In a further embodiment, an alkyl group has 1 to 4 carbon atoms (i.e., C₁, C₂, C₃, or C₄). Examples include methyl, ethyl, n-propyl, i-propyl, n-butyl, s-butyl, t-butyl, pentyl and hexyl, among others.

The term “cycloalkyl” is used herein to refer to cyclic, saturated aliphatic hydrocarbon groups. In one embodiment, a cycloalkyl group has 3 to 8 carbon atoms (i.e., C₃, C₄, C₅, C₆, C₇, or C₈). In another embodiment, a cycloalkyl group has 3 to 6 carbon atoms (i.e., C₃, C₄, C₅ or C₆). Examples include cyclopropyl, cyclobutyl, cyclopentyl and cyclohexyl, among others.

The term “alkenyl” is used herein to refer to both straight- and branched-chain alkyl groups having one or more carbon-carbon double bonds. In one embodiment, an alkenyl group contains 3 to 8 carbon atoms (i.e., C₃, C₄, C₅, C₆, C₇, or C₈). In another embodiment, an alkenyl group has 1 or 2 carbon-carbon double bonds and 3 to 6 carbon atoms (i.e., C₃, C₄, C₅ or C₆). Examples include propenyl, among others.

The term “alkynyl” is used herein to refer to both straight- and branched-chain alkyl groups having one or more carbon-carbon triple bonds. In one embodiment, an alkynyl group has 3 to 8 carbon atoms (i.e., C₃, C₄, C₅, C₆, C₇, or C₈). In another embodiment, an alkynyl group contains 1 or 2 carbon-carbon triple bonds and 3 to 6 carbon atoms (i.e., C₃, C₄, C₅, or C₆). Examples include propynyl, among others.

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents e.g. 1 to 3 substituents which may be the same or different, selected from hydrogen, halogen, CN, OH, NO₂, amino, aryl, heterocyclyl, aryl, alkoxy, aryloxy, alkylcarbonyl, alkylcarboxy, and arylthio. One suitable group of substituents is hydrogen, halogen, CN, OH, NO₂, amino, phenyl, C₁-C₄ alkoxy, phenoxy, C₁-C₄ alkylcarbonyl, C₁-C₄ alkylcarboxy and phenylthio.

The term “arylthio” as used herein refers to the S(aryl) group, where the point of attachment is through the sulfur-atom and the aryl group can be substituted, e.g., by 1 to 4 substituents, the same or different, selected from among hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio. The term “alkoxy” as used herein refers to the O(alkyl) group, where the point of attachment is through the oxygen-atom and the alkyl group can be substituted, e.g., by 1 to 4 substituents, the same or different, selected from among hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio. The term “aryloxy” as used herein refers to the O(aryl) group, where the point of attachment is through the oxygen-atom and the aryl group can be substituted, e.g., by 1 to 4 substituents, the same or different, selected from among hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio.

The term “alkylcarbonyl” as used herein refers to the C(O)(alkyl) group, where the point of attachment is through the carbon-atom of the carbonyl moiety and the alkyl group can be substituted, e.g., by 1 to 4 substituents, the same or different, selected from among hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio.

The term “alkylcarboxy” as used herein refers to the C(O)O(alkyl) group, where the point of attachment is through the carbon-atom of the carboxy moiety and the alkyl group can be substituted, e.g., by 1 to 4 substituents, the same or different, selected from among hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio.

The term “alkylamino” as used herein refers to both secondary and tertiary amines where the point of attachment is through the nitrogen-atom and the alkyl groups can be substituted, e.g., by 1 to 4 substituents, the same or different, selected from hydrogen, halogen, CN, OH, NO₂, amino, phenyl, C₁-C₄ alkyloxy, phenoxy, C₁-C₄ alkylcarbonyl, C₁-C₄ alkylcarboxyl and phenylthio. The alkyl groups can be the same or different.

The term “halogen” as used herein refers to Cl, Br, F, or I.

The term “aryl” as used herein refers to an aromatic, carbocyclic system, e.g., of 6 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituent selected from halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkoxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl. Desirably, a substituted aryl group is substituted with 1 to 4 substituents which may be the same or different.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms independently selected from among nitrogen, oxygen, and sulfur atoms. In one embodiment, the heterocyclic ring has 1 to 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of 6 to 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings. Suitable heterocyclic rings include those having 6 to 12, preferably 6 to 10 ring members containing 1 to 3 heteroatoms selected from N, O and S. Suitable heteroaryl rings include those having 5 to 12 preferably 5 to 10 ring members containing 1 to 3 heteroatoms independently selected from among N, O and S.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to 4 heteroatoms in the backbone of the ring which may suitably be independently selected from among O, S and N. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents, the same or different selected from halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkoxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl. A substituted heterocycle or heteroaryl group may have 1, 2, 3, or 4 substituents.

The compounds may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals.

Pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts may also be formed from inorganic bases, desirably alkali metal salts including, e.g., sodium, lithium, or potassium, and organic bases, such as ammonium salts, mono-, di-, and trimethylammonium, mono-, di- and triethylammonium, mono-, di- and tripropyl-ammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmonoethanolammonium, and the like.

Physiologically acceptable alkali salts and alkaline earth metal salts can include, without limitation, sodium, potassium, calcium and magnesium salts in the form of esters, and carbamates.

These salts, as well as other compounds, can be in the form of esters, carbamates and other conventional “pro-drug” forms, which, when administered in such form, convert to the active moiety in vivo. In one embodiment, the prodrugs are esters. In another embodiment, the prodrugs are carbamates. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

The compounds discussed herein also encompass “metabolites” which are unique products formed by processing the compounds by the cell or subject. Desirably, metabolites are formed in vivo.

### II. Methods of Preparation

The compounds described herein may be prepared by a number of novel routes. However, it is the specific combination of steps and reagents selected by the inventors that provide the compounds of formula (I), (Ia), or (Ib). In one embodiment, the compounds described herein may be prepared using the Heck reaction (Scheme 1) in which a compound of formula (II) is reacted with a vinyl compound of formula (III). R₁-R₆ and X are defined herein and LG is a leaving group.

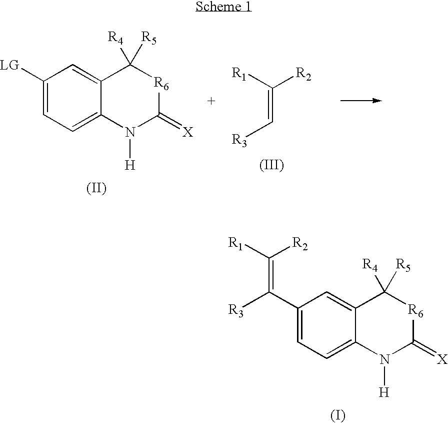

The term “leaving group” as used herein refers to a substituent that is present on a chemical compound and can be displaced (the term LG as used herein refers to a leaving group). The particular leaving group utilized is dependent upon the vinyl derivative (II) utilized and can readily be determined by one of skill in the art. Common leaving groups include, without limitation, halides and triflates. Desirably, the leaving group is a halide such as bromine, chlorine, or iodine. More desirably the leaving group is bromine.

The compounds of formula (II) may be prepared and purified using methods in the art and as described in U.S. Pat. Nos. 6,509,334; 6,436,929; 6,358,948; 6,444,668; US-2005/0239779; and US-2005/0250766, among others, which are hereby incorporated by reference.

The compounds of formula (III) may be purchased from commercial vendors including, without limitation, the Aldrich catalog, “Advancing Science”, Handbook of Fine Chemicals, 2007-2008, which is hereby incorporated by reference.

The reaction between compounds (II) and (III) is desirably performed in the presence of a catalyst. One of skill in the art would readily be able to select suitable catalyst for use in the reaction. Typically, the catalyst is a palladium catalyst. A variety of palladium catalysts may be selected and include, without limitation, dichloro[1,1′-bis(diphenylphosphino)ferrocene]palladium (II) dichloromethane adduct or tris(dibenzylideneacetone)dipalladium. Other catalysts are available in the art including those provided in March, Advanced Organic Chemistry, 4^{th} Ed.; John Wiley & Sons; New York, 1992, pp. 717-718; references cited in March et al.; and Heck, Org. React. 1982, 27, 345-390, which are hereby incorporated by reference.

The reaction is also desirably performed in the presence of a phosphine. One of skill in the art would readily be able to select a suitable phosphine for use in this reaction. A variety of phosphines are available from commercial vendors and catalogs such as the Aldrich catalog cited above and incorporated by reference and “Chemicals for Research”, the Strem catalog, No. 21, 2007-2008, which is hereby incorporated by reference. In one embodiment, the phosphine is triphenylphosphine, 1,3-bis(diphenylphosphino)propane, 1,2-bis(diphenylphosphino)ethane, 1,1′-bis(diphenylphosphino)ferrocene, or a tri-alkylphosphine(P(alkyl)₃) such as tri-n-butylphosphine or tri-tert-butylphosphine. In another embodiment, the phosphine is tri-tert-butylphosphine.

A weak base is also added to the reaction and may readily be selected by one of skill in the art. One of skill in the art would readily be able to select a suitable base for use in this reaction. A variety of bases are available from commercial vendors and catalogs such as the Aldrich catalog cited above and incorporated by reference. In one embodiment, the base is N,N-dicyclohexylmethylamine or potassium acetate, among others, including those discussed in Heck, Org. React. 1982, 27, 345-390, cited above and incorporated herein by reference.

The solvent for use in the reaction may also be selected by one of skill in the art. Desirably, the solvent is inert to the starting materials and reactants. A variety of solvents are available from commercial vendors and catalogs such as the Aldrich catalog cited above and incorporated by reference. In one embodiment, the solvent is dioxane, N,N-dimethylformamide, tetrahydrofuran, acetonitrile, methanol, N-methylpyrrolidinone hexamethylphosphoramide, or combinations thereof.

In one embodiment, the compounds of formula (I), (Ia), or (Ib) are prepared by reacting an appropriately substituted aryl halide (II) with a vinyl derivative (III), in a solvent such as dioxane or N,N-dimethylformamide containing a catalyst such as dichloro[1,1′-bis(diphenylphosphino)ferrocene]palladium (II) dichloromethane or tris(dibenzylideneacetone)dipalladium (0), in the presence of a base, with or without a tri-alkylphosphine, to give the desired aryl substituted vinyl derivatives (I). See, Scheme 2.

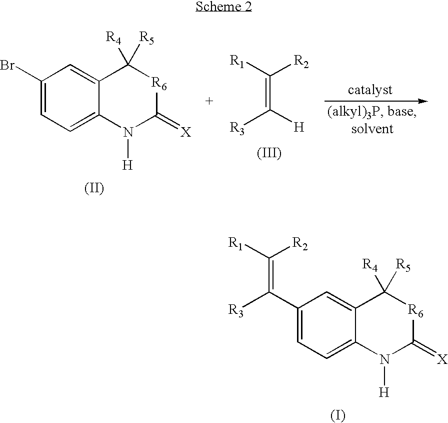

A second method of preparing compounds of formula (I) or (Ia), wherein either R₁ or R₂ is —CN, is described. This method includes reacting an amide of formula (IVa) or (IVb), wherein R₃-R₆ and X are defined herein and either R₁ or R₂ is CONH₂, with a dehydrating agent. See, Scheme 3.

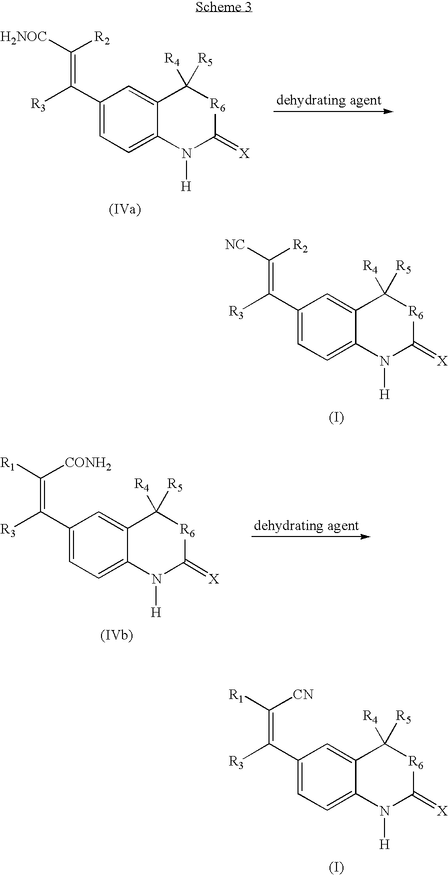

One of skill in the art can readily select a suitable dehydrating agent for use in this preparation. A variety of dehydrating agents are available from commercial vendors and catalogs such as the Aldrich catalog cited above and incorporated by reference. In one embodiment, the dehydrating agent is thionyl chloride, phosphorus oxychloride, phosphorus pentachloride, phosgene, oxalyl chloride, methanesulfonyl chloride or acetic anhydride. In another embodiment, the dehydrating agent is thionyl chloride.

The solvent for use in the dehydration reaction may also be selected by one of skill in the art. Desirably, the solvent is inert to the starting materials and reactants. A variety of solvents are available from commercial vendors and catalogs such as the Aldrich catalog cited above and incorporated by reference. In one embodiment, the solvent is dioxane, tetrahydrofuran, benzene, toluene, or chloroform, among others.

The reaction temperature of the dehydration may be determined by one of skill in the art based on the reagents utilized therein. In one embodiment, the dehydration is performed at room temperatures. In another embodiment, the dehydration is performed at elevated temperatures. In a further embodiment, the dehydration is performed at temperatures of about room temperature to the refluxing temperature of the solvent being used. In still another embodiment, the dehydration is performed at about 75° C.

In one embodiment, compounds of formula (I) or (Ia) wherein either R₁ or R₂ is CN may be prepared according to Scheme 4. In this method, the appropriate amide (IVa) or (IVb), wherein either R₁ or R₂ is CONH₂, is heated in a solvent such as dioxane and in the presence of a dehydrating reagent such as thionyl chloride to provide the desired vinyl nitrile (I).

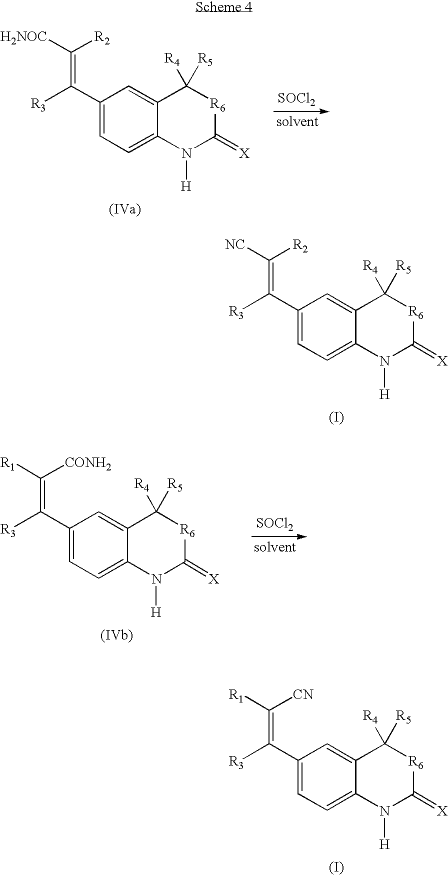

### III. Methods of Using the Compounds

Also provided are pharmaceutical compositions containing one or more compounds described herein and a pharmaceutically acceptable carrier or excipient. In one embodiment, the methods of treatment include administering to a mammal a pharmaceutically effective amount of one or more compounds as described herein as progesterone receptor modulators.

The compounds may be combined with one or more pharmaceutically acceptable carriers or excipients, e.g., solvents, diluents and the like. Suitably, the compounds are formulated for delivery to a subject by any suitable route including, e.g., transdermal, mucosal (intranasal, buccal, vaginal), oral, parenteral, among others. A variety of suitable delivery devices can be utilized for these delivery routes and include, without limitation, tablets, caplets, capsules, gel tabs, dispersible powders, granules, suspensions, injectable solutions, transdermal patches, topical creams or gels, and vaginal rings, among others.

In preparing the compositions described herein, the compounds may be combined with one or more of a solid carrier, liquid carrier, adjuvant, suspending agent, syrup, and elixir, among others, the selection of which is dependent on the nature of the active ingredient and the particular form of administration desired.

Solid carriers include, without limitation, starch, lactose, dicalcium phosphate, microcrystalline cellulose, sucrose and kaolin.

Liquid carriers include, without limitation, sterile water, dimethylsulfoxide (DMSO), polyethylene glycols, non-ionic surfactants and edible oils such as corn, peanut and sesame oils.

Adjuvants can include, without limitation, flavoring agents, coloring agents, preserving agents, and antioxidants, e.g., vitamin E, ascorbic acid, butylatedhydroxytoluene (BHT) and butylatedhydroxyanisole (BHA).

In one embodiment, the compound may be combined with a suspending agent, including about 0.05 to about 5% by weight of suspending agent. In another embodiment, the compound may be combined with a syrup containing, e.g., about 10 to about 50% by weight of sugar including, without limitation, sucrose or fructose. In a further embodiment, the compound may be combined with an elixir containing, e.g., about 20 to about 50% by weight ethanol, and the like.

When formulated for oral delivery, the compounds can be in the form of a tablet, capsule, caplet, gel tab, dispersible powder, granule, or suspension. One particularly desirable pharmaceutical composition, from the standpoint of ease of preparation and administration, are solid compositions, particularly tablets and hard-filled or liquid-filled capsules.

The compounds may also be administered parenterally or intraperitoneally as solutions, suspensions, dispersions, or the like. Such pharmaceutical preparations may contain, e.g., about 25 to about 90% by weight of the compound in combination with the carrier. Desirably, the pharmaceutical preparation contains about 5% and 60% by weight of the compound. In one embodiment, the compounds are administered in solutions or suspensions, whereby the compounds are present as free bases or pharmacologically acceptable salts and are prepared in water suitably mixed with a surfactant such as hydroxypropylcellulose. In another embodiment, the solutions or suspensions containing the compound may contain about 0.05 to about 5% by weight of a suspending agent in an isotonic medium. In a further embodiment, the compounds are administered in dispersions, which may be prepared in glycerol, polyethylene glycols and mixtures thereof in oils.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacterial and fungi. The carrier utilized in the injectable form may be a solvent or dispersion medium containing, e.g., water, ethanol (e.g., glycerol, propylene glycol and liquid polyethylene glycol), suitable mixtures thereof, and vegetable oil.

The compounds may also be administered via a vaginal ring. Suitably, use of the vaginal ring is timed to cycle to which the compound is being administered, including a 28-day cycle. However, the vaginal ring can be inserted for longer or shorter periods of time. See, U.S. Pat. Nos. 5,972,372; 6,126,958; and 6,125,850, which are hereby incorporated by reference, for formulations of the vaginal ring that can be used.

The compounds can also be delivered via a transdermal patch. Suitably, use of the patch is timed to the length of the cycle, including a 28 day cycle. However, the patch can remain in place for longer or shorter periods of time.

The compounds may be utilized in methods of contraception, hormone replacement therapy, stimulating food intake, and the treatment and/or prevention of benign and malignant neoplastic disease; cycle-related symptoms; fibroids, including uterine fibroids; leiomyoma; endometriosis; polycystic ovary syndrome; benign prostatic hypertrophy; hormone-dependent carcinomas and adenocarcinomas of the endometrium, ovary, breast, colon, prostate, pituitary, meningioma and other hormone-dependent carcinomas; dysmenorrhea; menopause related symptoms; dysfunctional uterine bleeding; symptoms of premenstrual syndrome and premenstrual dysphoric disorder; and for inducing amenorrhea. Additional uses of the present progesterone receptor modulators include the synchronization of estrus in livestock. In one embodiment, the neoplastic disease is hormone-dependent.

The term “cycle-related symptoms” refers to psychological symptoms (e.g., mood change, irritability, anxiety, lack of concentration, or decrease in sexual desire) and physical symptoms (e.g., dysmenorrhea, breast tenderness, bloating, fatigue, or food cravings) associated with a woman's menstrual cycle. Cycle-related symptoms include, but are not limited to, dysmenorrhea and moderate to severe cycle-related symptoms.

When utilized for these purposes, the compounds can be administered in combination with other agents, as well as in combination with each other. Such agents include, without limitation, progestins, antiprogestins, estrogens, antiestrogens, selective estrogen receptor modulators (SERMS), among others. Progestins can include, without limitation, tanaproget, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrot, (17-deacetyl)norgestimate. Estrogens can include, without limitation, ethinyl estradiol. The compounds described herein can be combined with one or more of these agents, delivered concurrently therewith one or more of these agents, delivered prior to one or more of these agents, or delivered subsequent to one or more of these agents.

A patient or subject being treated is a mammalian subject and typically a female. Desirably, the subject is a human. However, as used herein, a female can include non-human mammals, e.g., cattle or livestock, horses, pigs, domestic animals, etc.

The effective dosage of the compound may vary depending on the particular compound employed, the mode of administration and the severity of the condition being treated. However, in general, satisfactory results are obtained when the compounds are administered at a daily dosage of about 0.5 to about 500 mg/kg of animal body weight, about 1 to about 400 mg/kg, about 5 to about 300 mg/kg, about 10 to about 250 mg/kg, about 50 to about 200 mg/kg, or about 100 to 150 mg/kg. For most large mammals, the total daily dosage is from about 1 to 100 mg. In one embodiment, the total daily dosage is from about 2 to 80 mg. This dosage regimen may be adjusted to provide the optimal therapeutic response. For example, several divided doses may be administered daily or the dose may be proportionally reduced as indicated by the exigencies of the therapeutic situation.

As previously noted, the compounds may be administered via a vaginal ring. In one embodiment, the ring is inserted into the vagina and it remains in place for 3 weeks. During the fourth week, the vaginal ring is removed and menses occurs. The following week, a new ring is inserted to be worn another 3 weeks until it is time for the next period. In another embodiment, the vaginal ring is inserted weekly and is replaced for 3 consecutive weeks. Then, following 1 week without the ring, a new ring is inserted to begin a new regimen. In yet another embodiment, the vaginal ring is inserted for longer or shorter periods of time.

Further, the previously mentioned transdermal patch is applied via a suitable adhesive on the skin, where it remains in place for at least one week. In one embodiment, the transdermal patch remains in place for one week and is replaced weekly for a total of 3 weeks. In another embodiment, the transdermal patch remains in place for two weeks. In a further embodiment, the transdermal patch remains in place for three weeks. During the fourth week, no patch is applied and menses occurs. The following week, a new patch is applied to be worn to begin a new regimen. In yet another embodiment, the patch remains in place for longer or shorter periods of time.

When used for contraception, the method typically includes delivering a daily dosage unit containing a compound for 28 consecutive days to a female of child-bearing age. Desirably, the method includes delivering the compound over a period of 21 to 27 consecutive days followed by 1 to 7 consecutive days in which no effective amount or no amount of the compound is delivered. Optionally, the period of 1 to 7 days in which no effective amount of the compound is delivered to the subject can involve delivery of a second phase of daily dosage units of 1 to 7 days of a pharmaceutically acceptable placebo. Alternatively, during this “placebo period”, no placebo is administered. The compound may optionally be administered in combination with a progestin, antiprogestin, estrogen, or combination thereof.

In another embodiment, the method includes delivering a compound for 21 consecutive days, followed by 7 days in which no effective amount of the compound is delivered. Optionally, during these 7 days, a second phase of 7 daily dosage units of an orally and pharmaceutically acceptable placebo can be delivered. The compound may optionally be administered in combination with a progestin, antiprogestin, estrogen, anti-estrogen, SERM or combination thereof.

In a further embodiment, the method includes delivering a compound for 23 consecutive days, followed by 5 days in which no effective amount of the compound is delivered. Optionally, during these 5 days, a second phase of 5 daily dosage units of an orally and pharmaceutically acceptable placebo can be delivered. The compound may optionally be administered in combination with a progestin, antiprogestin, estrogen, anti-estrogen, SERM or combination thereof.

In yet another embodiment, the method includes delivering a compound for 25 consecutive days, followed by 3 days in which no effective amount of the compound is delivered. Optionally, during these 3 days, a second phase of 3 daily dosage units of an orally and pharmaceutically acceptable placebo can be delivered. The compound may optionally be administered in combination with a progestin, antiprogestin, estrogen anti-estrogen, SERM, or combination thereof.

In still a further embodiment, the method includes delivering a compound for 27 consecutive days, followed by 1 day in which no effective amount of the compound is delivered. Optionally, a second phase of 1 daily dosage unit of an orally and pharmaceutically acceptable placebo can be delivered. The compound may optionally be administered in combination with a progestin, antiprogestin, estrogen, anti-estrogen, SERM, or combination thereof.

In another embodiment, a method of contraception includes administering to a female of child bearing age for 28 consecutive days: (a) a first phase of from 14 to 24 daily dosage units of a progestational agent equal in progestational activity to about 35 to about 100 μg levonorgestrel; (b) a second phase of from 1 to 11 daily dosage units, at a daily dosage of from about 2 to 50 mg, of a compound described herein; and (c) optionally, a third phase of daily dosage units of an orally and pharmaceutically acceptable placebo for the remaining days of the 28 consecutive days in which no antiprogestin, progestin or estrogen is administered; wherein the total daily dosage units of the first, second and third phases equals 28.

In yet a further embodiment, a method of contraception includes administering to a female of child bearing age for 28 consecutive days: (a) a first phase of from 14 to 24 daily dosage units of a compound described herein; (b) a second phase of from 1 to 11 daily dosage units of an antiprogestin; and (c) optionally, a third phase of daily dosage units of an orally and pharmaceutically acceptable placebo for the remaining days of the 28 consecutive days in which no antiprogestin, progestin, estrogen, anti-estrogen or SERM is administered; wherein the total daily dosage units of the first, second and third phases equals 28.

In yet a further embodiment, a method of contraception is provided and includes administering to a female of child bearing age for 28 consecutive days: (a) a first phase of from 14 to 24 daily dosage units of a progestational agent equal in progestational activity to about 35 to about 100 μg levonorgestrel; (b) a second phase of from 1 to 11 daily dosage units, at a daily dosage of from about 2 to 50 mg, of a compound described herein; and (c) optionally, a third phase of daily dosage units of an orally and pharmaceutically acceptable placebo for the remaining days of the 28 consecutive days in which no antiprogestin, progestin or estrogen is administered; wherein the total daily dosage units of the first, second and third phases equals 28.

In another embodiment, a method of contraception is provided and includes administering to a female of child bearing age for 28 consecutive days: (a) a first phase of from 14 to 24 daily dosage units of a compound described herein; (b) a second phase of from 1 to 11 daily dosage units of an antiprogestin; and (c) optionally, a third phase of daily dosage units of an orally and pharmaceutically acceptable placebo for the remaining days of the 28 consecutive days in which no antiprogestin, progestin, estrogen, anti-estrogen or SERM is administered; wherein the total daily dosage units of the first, second and third phases equals 28.

Also provided are kits or packages of pharmaceutical formulations designed for use in the regimens described herein. Suitably, the kits contain one or more compounds as described herein.

Advantageously, for use in the kits, the compound is formulated for the desired delivery vehicle and route. For example, the compound can be formulated for oral delivery, parenteral delivery, vaginal ring, transdermal delivery, or mucosal delivery, as discussed in detail above. The kit is preferably a pack (e.g. a blister pack) containing daily doses arranged in the order in which they are to be taken.

In each of the regimens and kits described herein, it is preferred that the daily dosage of each pharmaceutically active component of the regimen remain fixed in each particular phase in which it is administered. It is also understood that the daily dose units described are to be administered in the order described, with the first phase followed in order by the optional phases, including any second and third phases. To help facilitate compliance with each regimen, it is also preferred that the kits contain the placebo described for the final days of the cycle. It is further preferred that each package or kit contain a pharmaceutically acceptable package having indicators for each day of the 28-day cycle, such as a labeled blister package, dial dispenser, or other packages known in the art.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units which are administered over the course of each day of the cycle contemplated.

In one embodiment, the kit is designed for daily oral administration over a 28-day cycle, desirably for one oral administration per day, and organized so as to indicate a single oral formulation or combination of oral formulations to be taken on each day of the 28-day cycle. Desirably each kit will include oral tablets to be taken on each the days specified; desirably one oral tablet will contain each of the combined daily dosages indicated. For example, a kit can contain 21 to 27 daily dosage units of an effective amount of the compound, optionally, 1 to 7 daily dosage units of a placebo and other appropriate components including, e.g., instructions for use.

In another embodiment, the kit is designed for weekly or monthly administration via a vaginal ring over a 28-day cycle. Suitably, such a kit contains individual packaging for each of the vaginal rings, i.e. one to three, required for a monthly cycle and other appropriate components, including, e.g., instructions for use.

In a further embodiment, the kit is designed for weekly or monthly administration via a transdermal patch over a 28-day cycle. Suitably, such a kit contains individual packaging for each of the patches, i.e. one to three, required for a monthly cycle and other appropriate components including, e.g., instructions for use.

In still another embodiment, the kit is designed for parenteral delivery of the compound. Such a kit is typically designed for delivery at home and may include needles, syringes, and other appropriate packaging and instructions for use.

In yet another embodiment, the kit contains the compound in a gel or cream formulation. Optionally, the kit can include appropriate packaging such as a tube or other container, an applicator, and/or instructions for use.

In a further embodiment, the kit includes (a) a first phase of from 14 to 21 daily dosage units of a progestational agent equal in progestational activity to about 35 to about 150 pg levonorgestrel; (b) a second phase of from 1 to 11 daily dosage units of a compound described herein; and (cc) a third phase of daily dosage units of an orally and pharmaceutically acceptable placebo; wherein the total number of the daily dosage units in the first phase, second phase and third phase equals 28.

In still another embodiment, a kit contains (a) a first phase of from 14 to 21 daily dosage units of a compound described herein; (b) a second phase of from 1 to 11 daily dosage units of an antiprogestin compound; and (c) a third phase of daily dosage units of an orally and pharmaceutically acceptable placebo; wherein the total number of the daily dosage units in the first phase, second phase and third phase equals 28.

The following examples are illustrative only and are not intended to be a limitation on the present invention.

### EXAMPLES

### Example 1

### Methyl(2E)-3-(4,4-dimethyl-2-oxo-1,2,3,4-tetrahydroquinolin-6-yl)acrylate

Step 1: N-(4-Bromophenyl)-3-methylbut-3-enamide

3-Methylbut-2-enoyl chloride (16.2 mL, 146 mmol) in methylene chloride (50 mL) was added under nitrogen drop-wise over 1 hour to a solution of 4-bromoaniline (25.04 g, 146 mmol) and triethylamine (24.4 mL, 175 mmol) in methylene chloride (500 mL) at room temperature. After the addition, the reaction was stirred at room temperature for 23 hours (overnight). The reaction was extracted with 2 N HCl, 5% NaHCO₃, dried (anhydrous MgSO₄), filtered and the solvent removed under reduced pressure to give a light brown solid (35.47 g). Recrystallization of the solid from isopropyl alcohol gave N-(4-bromophenyl)-3-methylbut-3-enamide (6.2082, 17%) as a white solid, mp 109-112° C., MS (ESI) m/z 254; Anal. Calcd. for C₁₁H₁₂BrNO: C, 51.99; H, 4.76; N, 5.51. Found: C, 51.86; H, 4.73; N, 5.41.

The mother liquor from the recrystallization was concentrated under reduced pressure to give 28.74 g of a brown solid. NMR analysis of this solid showed it to be an approximately two to one mixture of N-(4-bromophenyl)-3-methylbut-3-enamide and N-(4-bromophenyl)-3-methylbut-2-enamide.

Step 2: 6-Bromo-4,4-dimethyl-3,4-dihydroquinolin-2(1H)-one

Aluminum chloride (15.95 g, 119.6 mmol) was added under nitrogen to a mixture of N-(4-bromophenyl)-3-methylbut-3-enamide and N-(4-bromophenyl)-3-methylbut-2-enamide (10.11 g, 39.8 mmol), prepared in the previous step, in methylene chloride (500 mL). After the addition, the reaction was refluxed for 4 hours. After cooling to room temperature, 2 N HCl was added drop-wise. The reaction was partitioned with 2 N HCl. The aqueous layer was separated and extracted three times with methylene chloride. The combined organic extracts were dried (anhydrous MgSO₄), filtered and the solvent removed under reduced pressure to give a brown solid (10.12 g). Recrystallization of the solid from isopropyl alcohol gave 6-bromo-4,4-dimethyl-3,4-dihydroquinolin-2(1H)-one (3.6570 g, 36%) as a white solid, mp 176-179° C., MS (ESI) m/z 254; Anal. Calcd. for C₁₁H₁₂BrNO: C, 51.99; H, 4.76; N, 5.51. Found: C, 51.86; H, 4.53; N, 5.38.

Step 3: Methyl(2E)-3-(4,4-dimethyl-2-oxo-1,2,3,4-tetrahydroquinolin-6-yl)acrylate

A mixture of 6-bromo-4,4-dimethyl-3,4-dihydroquinolin-2(1H)-one (250 mg, 0.984 mmol), prepared in the previous step, methyl acrylate (97 μL, 1.08 mmol) and potassium acetate (106 mg, 1.08 mmol) in anhydrous N,N-dimethylformamide (5 mL) was purged with nitrogen. Dichloro[1,1′-bis(diphenylphosphino)ferrocene]palladium (II) dichloromethane adduct (40 mg, 0.049 mmol) was added and the mixture stirred under nitrogen at 95° C. for 4 hours and then overnight at room temperature. By liquid chromatography (LC)/mass spectroscopy (MS) analysis, the reaction was not complete. An additional 1.5 equivalents of methyl acrylate was added and the mixture purged with nitrogen. Additional dichloro[1,1′-bis(diphenylphosphino)ferrocene]palladium (II) dichloromethane adduct was added and the mixture stirred at 95° C. for 5 hours. The reaction was partitioned between ethyl acetate and water. The organic layer was separated, washed three times with water, dried (anhydrous MgSO₄), filtered and the solvent removed under reduced pressure to give 224 mg of a thick oil. Purification of the oil on a Horizon™ Flash 25+™ M silicon column (Biotage) using a linear gradient of 26% ethyl acetate-hexane to 37% ethyl acetate-hexane as the eluents gave the title compound (85 mg, 33%) as a white solid, mp 174-175° C.; MS (ESI) m/z 260; Anal. Calcd. for C₁₅H₁₇NO₃: C, 69.48; H, 6.61; N, 5.40. Found: C, 69.21; H, 6.76; N, 5.27.

### Example 2

### Methyl(2E)-3-(4,4-dimethyl-2-oxo-1,4-dihydro-2H-3,1-benzoxazin-6-yl)acrylate

Step 1: 2-(2-Amino-5-bromo-phenyl)-propan-2-ol

This compound was prepared as described in U.S. Pat. No. 6,444,668. A solution of 2-amino-5-bromobenzoic acid (10 g, 46 mmol) in dry tetrahydrofuran (THF; 200 mL) was treated at −78° C. under nitrogen with a solution of methylmagnesium bromide in ether (3.0 M, 90 mL, 270 mmol). The reaction was slowly warmed to ambient temperature, kept stirring for 48 hours under nitrogen and then poured into cold 0.5 N HCl (300 mL). The mixture was neutralized with 1 N NaOH and ethyl acetate (300 mL) was added. The organic layer was separated and the aqueous layer was extracted with ethyl acetate (3×100 mL). The combined organic layers were washed with brine, dried over anhydrous MgSO₄ and filtered. After removal of the solvent under reduced pressure, the residue was purified by silica gel flash chromatography (hexane:ethyl acetate/3:2) to give 2-(2-amino-5-bromo-phenyl)-propan-2-ol (6 g, 57%) as an off-white solid, mp 62-63 ° C.

Step 2: 6-Bromo-4,4-dimethyl-1,4-dihydro-2H-3,1-benzoxazin-2-one

This compound was prepared as described in U.S. Pat. No. 6,444,668. To a solution of 2-(2-amino-5-bromo-phenyl)-propan-2-ol (18 g, 78 mmol), prepared in the previous step, in dry THF (150 mL) was added 1,1′-carbonyldiimidazole (15.5 g, 94 mmol) under nitrogen. The reaction was heated at 50° C. overnight. The solvent was removed under reduced pressure and the residue was dissolved in ethyl acetate (100 mL). The solution was washed with 1 N HCl (2×40 mL), brine (20 mL), dried over anhydrous MgSO₄ and filtered. After removal of the solvent under reduced pressure 6-bromo-4,4-dimethyl-1,4-dihydro-2H-3,1-benzoxazin-2-one (20 g, 100%) was obtained as a white solid, mp 199-200° C.

Step 3: Methyl(2E)-3-(4,4-dimethyl-2-oxo-1,4-dihydro-2H-3,1-benzoxazin-6-yl)acrylate

A mixture of 6-bromo-4,4-dimethyl-1,4-dihydro-2H-3,1-benzoxazin-2-one (2.56 g, 10 mmol), prepared in the previous step, and tris(dibenzylideneacetone)dipalladium (0) (275 mg, 0.3 mmol) was purged with nitrogen. To this mixture was added methyl acrylate, (990 μL, 11 mmol), tri-tert-butylphosphine (1.86 mL, 10% by weight in hexane; 0.6 mmol), N,N-dicyclohexylmethylamine (2.33 mL, 11 mmol) and 10 mL of dioxane, in that order. The reaction was again purged with nitrogen and then stirred at room temperature. After a few hours, an LC/MS indicated that the starting material was gone. The reaction was dissolved in ethyl acetate and filtered through silica gel. The filtrate was concentrated under reduced pressure to give a yellow solid (3.1 g). Purification of the solid on a Horizon™ Flash Collector (Biotage Si column) using a linear gradient of 5% ethyl acetate-hexane to 50% ethyl acetate-hexane gave a light yellow solid (2.14 g). By thin layer chromatography (TLC) analysis the material was not pure. The solid was again purified on a Horizon™ Flash™ silicon column (Biotage), this time using a linear gradient of 5% ethyl acetate-methylene chloride to 20% ethyl acetate-methylene chloride as the eluent. After removal of the solvent under reduced pressure, the title compound (2.14 g, 82%) was isolated as a white solid, mp 204-205° C., MS (ESI) m/z 262, MS (ESI) m/z 260; Anal. Calcd. for C₁₄H₁₅NO₄: C, 64.36; H, 5.79; N, 5.36. Found: C, 64.25; H, 5.99; N, 5.25.

### Example 3

### 6-[(E)-2-Isoxazol-5-ylvinyl]-4,4-di-methyl-1,4-dihydro-2H-3,1-benzoxazin-2-one

n-Butyl lithium (2.01 mL of a 2.5 M solution in hexanes; 5.024 mmol) was added under nitrogen to a solution of acetaldehyde oxime (117 μL, 1.915 mmol) in anhydrous tetrahydrofuran (4 mL) at 0° C. After the addition, the cooling bath was removed and the stirring continued for 30 minutes. Methyl(2E)-3-(4,4-dimethyl-2-oxo-1,4-dihydro-2H-3,1-benzoxazin-6-yl)acrylate (250 mg, 0.957 mmol), prepared in step 3 of Example 2, was added to anhydrous tetrahydrofuran (1 mL). The solid did not completely dissolve. The supernatant liquid was added to the reaction. This process was repeated two additional times using anhydrous tetrahydrofuran (2 mL). After the last addition, the reaction was allowed to stir for approximately 2 hours. With caution, concentrated H₂SO₄ (1.25 mL) was added. The mixture was stirred for 15 minutes. 2 N NaOH was added to bring the mixture to a pH=7. The organic layer was separated and the aqueous layer was extracted with methylene chloride. The combined organic extracts were washed one time with water, dried (anhydrous MgSO₄), filtered and the solvent removed under reduced pressure to give a yellow oil (300 mg). Purification of the oil on a Horizon™ Flash™ silicon column (Biotage) using a linear gradient of 15% ethyl acetate-methylene chloride-30% ethyl acetate-methylene chloride gave an off-white solid (70 mg). By TLC analysis, the material was not pure. The solid was again purified on a Horizon™ Flash™ silicon column (Biotage), this time using a linear gradient of 5% ethyl acetate-hexane to 20% ethyl acetate-hexane as the eluent. After removal of the solvent under reduced pressure, the title compound (60 mg, 23%) was isolated as a white solid, mp 190-193° C., MS (ESI) m/z 271, MS (ESI) m/z 269; Anal. Calcd. for C₁₅H₁₄N₂O₃: C, 66.66; H, 5.22; N, 10.36. Found: C, 66.70; H, 5.45; N, 9.79.

### Example 4

### (2E)-3-(4,4-Dimethyl-2-oxo-1,4-dihydro-2H-3,1-benzoxazin-6-yl)acrylamide

A mixture 6-bromo-4,4-dimethyl-1,4-dihydro-2H-3,1-benzoxazin-2-one (1.0 g, 3.90 mmol), prepared in step 2 of Example 2, acrylamide (305 mg, 4.295 mmol), N,N-dicyclohexylmethylamine (0.91 mL, 4.295 mmol) and tri-tert-butylphosphine (52.6 mg of 90% technical grade; 0.234 mmol) in dioxane (5 mL) was purged with nitrogen. Tris(dibenzylideneacetone)dipalladium (0) (107 mg, 0.117 mmol) was added and the mixture again purged with nitrogen and then stirred under nitrogen at room temperature. After approximately 1.5 hours, the reaction had become a solid mass. Ethyl acetate was added, the solids were broken up and then stirred for 5 minutes. This mixture was filtered through silica gel. The silica gel was rinsed with ethyl acetate and the combined filtrates concentrated under reduced pressure to give an orange oil (500 mg). Analysis of the oil indicated that it was mostly dibenzylideneacetone. The silica gel was also rinsed with methanol. The methanol solution was concentrated under reduced pressure to give an off-white solid. Recrystallization of the solid from isopropyl alcohol gave the title compound (0.55 g, 57%) as an off-white solid, mp 196-198° C., MS (ESI) m/z 247, MS (ESI) m/z 245; Anal. Caled. for C₁₃H₁₄N₂O₃. 0.29 C₃H₈O: C, 63.18; H, 6.24; N, 10.62. Found: C, 63.03; H, 6.45; N, 10.28.

### Example 5

### (2E)-3-(4,4-Dimethyl-2-oxo-1,4-dihydro-2H-3,1-benzoxazin-6-yl)but-2-enamide

The reaction was set up in the same manner as described in Example 4, replacing acrylamide with (E)-but-2-enamide (366 mg, 4.295 mmol). The reaction was stirred overnight at room temperature and then filtered through a small amount of silica gel. The silica gel was rinsed with ethyl acetate and methanol. The filtrates were combined and the solvent removed under reduced pressure. Purification of the residue on a Horizon™ Flash™ silicon column (Biotage) using a linear gradient of 1% methanol-methylene chloride-5% methanol-methylene chloride gave the title compound (0.8 g, 79%) as a white solid, mp 198-200° C., MS (ESI) m/z 261, MS (ESI) m/z 259; Anal. Caled. for C₁₄H₁₆N₂O₃. 0.10 CH₂Cl₂: C, 63.01; H, 6.08; N, 10.42. Found: C, 62.65; H, 6.39; N, 10.15.

### Example 6

### (2E)-3-(4,4-Dimethyl-2-oxo-1,4-dihydro-2H-3,1-benzoxazin-6-yl)-3-phenylacrylonitrile

The reaction was set up in the same manner as described in Example 4, replacing acrylamide with trans-cinnamonitrile (540 μL, 4.295 mmol). The reaction was stirred at room temperature over the weekend and then filtered through a small amount of silica gel. The silica gel was rinsed with ethyl acetate and the filtrate concentrated under reduced pressure. The residue was purified on a Horizon™ Flash™ silicon column (Biotage) using a linear gradient of 15% ethyl acetate-methylene chloride-25% ethyl acetate-methylene chloride as the eluents. By analytical high performance liquid chromatography (HPLC), the material was not pure. Additional purification by reverse phase chromatography using a Luna® C8(2) 21.2×250 mm column using a gradient of 50% water/50% acetonitrile to 30% water/70% acetonitrile gave the title compound (190 mg, 16%) as a white solid, mp 205-206° C., MS (ESI) m/z 305, MS (ESI) m/z 303; Anal. Caled. for C₁₉H₁₆N₂O₂. 0.08 CH₂Cl₂: C, 73.65; H, 5.24; N, 9.00. Found: C, 72.84; H, 4.99; N, 8.66.

### Example 7

### (2E)-3-(4,4-Dimethyl-2-oxo-1,4-dihydro-2H-3,1-benzoxazin-6-yl)-3-phenylacrylamide

The reaction was set up in the same manner as described in Example 4, replacing acrylamide with trans-cinnamamide (606 mg, 4.295 mmol). The reaction was stirred overnight at room temperature and then filtered through a small amount of silica gel. The silica gel was rinsed with ethyl acetate and methanol and the filtrates concentrated under reduced pressure. Purification of the residue on a Horizon™ Flash™ silicon column (Biotage) gave the title compound (700 mg, 56%) as a white solid, mp 219-220° C., MS (ESI) m/z 323, MS (ESI) m/z 321; Anal. Calcd. for C₁₉H₁₈N₂O₃. 0.56 C₃H₈O: C, 69.77; H, 6.36; N, 7.87. Found: C, 67.22; H, 6.90; N, 7.45.

### Example 8

### (2E)-3-(4,4-Dimethyl-2-oxo-1,4-dihydro-2H-3,1-benzoxazin-6-yl)acrylonitrile

The reaction was set un in the same manner as described in Example 4, replacing acrylamide with acrylonitrile (283 μL, 4.295 mmol). The reaction was stirred at room temperature overnight and then filtered through a small amount of silica gel. The silica gel was rinsed with ethyl acetate and then with methanol and the filtrate concentrated under reduced pressure. The residue was purified on a Horizon™ Flash™ silicon column (Biotage) using a linear gradient of 15% ethyl acetate-methylene chloride-25% ethyl acetate-methylene chloride as the eluents. By analytical HPLC, the material was not pure. Additional purification by reverse phase chromatography using a Luna® C18(2) 50×250 mm column and a gradient of 90% water/10% acetonitrile to 5% water/95% acetonitrile gave the title compound (46 mg, 51%) as a white solid, mp 238-240° C. (dec), MS (ESI) m/z 229, MS (ESI) m/z 227; Anal. Calcd. for 1.00 C₁₃H₁₂N₂O₂+0.03 CH₂Cl₂: C, 68.41; H. 5.30; N, 12.27. Found: C, 67.38; H, 4.91; N, 12.14.

### Example 9

### Methyl(2E)-3-(4,4-dimethyl-2-oxo-1,4-dihydro-2H-3,1-benzoxazin-6-yl)but-2-enoate

In the same manner as described in Example 8, replacing acrylonitrile with methyl crotonate (456 μL, 4.295 mmol), the title compound (47 mg, 44%) was isolated as a white solid, mp 131-132° C., MS (ESI) m/z 274; Anal. Calcd. for C₁₅H₁₇NO₄: C, 65.44; H, 6.22; N, 5.09. Found: C, 65.38; H, 6.33; N, 4.99

### Example 10

### 4,4-Dimethyl-6-(6-oxo-3,6-dihydro-2H-pyran-4-yl)-1,4-dihydro-2H-3,1-benzoxazin-2-one

The reaction was set up in the same manner as described in Example 4, replacing acrylamide with 5,6-dihydro-2H-pyran-2-one (370 μL, 4.295 mmol). The reaction was stirred at room temperature overnight and then filtered through a small amount of silica gel. The silica gel was rinsed with ethyl acetate and the filtrate concentrated under reduced pressure to remove the solvent. The residue was purified on a Horizon™ Flash™ silicon column (Biotage) to give the title compound (0.23 g, 21%) as a white solid, mp 235-236° C. (dec)3 MS (ESI) m/z 272; Anal. Calcd. for 1.00 C₁₅H₁₅NO₄+0.05 CH₂Cl₂: C, 65.92; H, 5.53; N, 5.13. Found: C, 65.13; H, 5.58; N, 4.97.

### Example 11

### Methyl(2E)-3-(4,4-dimethyl-2-oxo-1,4-dihydro-2H-3,1-benzoxazin-6-yl)-3-phenylacrylate

The reaction was set up in the same manner as described in Example 4, replacing acrylamide with trans-methyl cinnamate (696 mg, 4.295 mmol). The reaction was stirred at room temperature overnight and then filtered through a small amount of silica gel. The silica gel was rinsed with ethyl acetate and the filtrate concentrated under reduced pressure. The residue was purified on a Horizon™ Flash™ silicon column (Biotage) using a linear gradient of 5% ethyl acetate-methylene chloride-15% ethyl acetate-methylene chloride as the eluents. By analytical HPLC, the material was not pure. Additional purification by reverse phase chromatography using a Luna® C8(2) 21.2×250 mm column and a gradient of 50% water/50% acetonitrile to 0% water/100% acetonitrile gave the title compound (225 mg, 17%) as a white solid, mp 181-182° C., MS (ESI) m/z 336; Anal. Calcd. for 1.00 C₂₀H₁₉NO₄+0.05 CH₂Cl₂: C, 71.20; H, 5.68; N, 4.15. Found: C, 70.61; H, 5.61; N, 4.02.

### Example 12

### (2Z)-3-(4,4-Dimethyl-2-oxo-1,4-dihydro-2H-3,1-benzoxazin-6-yl)but-2-enenitrile

The reaction was set up in the same manner as described in Example 4, replacing acrylamide with crotononitrile (350 μL, 4.295 mmol). The reaction was stirred at room temperature overnight and then filtered through a small amount of silica gel. The silica gel was rinsed with ethyl acetate and the filtrate concentrated under reduced pressure. Purification of the residue by reverse phase chromatography using a Luna® C18(2) 50×250 mm column and a gradient of 65% water/35% acetonitrile to 0% water/100% acetonitrile gave the title compound (80 mg, 8%) as a white solid, mp 213-215° C., MS (ESI) m/z 243 Anal. Calcd. for C₁₄H₁₄N₂O₂. 0.12 CH₂Cl₂: C, 67.17; H. 5.69; N, 11.10. Found: C, 66.20; H, 5.59; N, 10.78.

### Example 13

### (2E)-3-(4,4-Dimethyl-2-oxo-1,4-dihydro-2H-3,1-benzoxazin-6-yl)but-2-enenitrile

Using the exact conditions as described in Example 12, the title compound (100 mg, 11%) was isolated as a white solid, mp 230-232° C., MS (ESI) m/z 243, MS (ESI) m/z 241; Anal. Calcd. for C₁₄H₁₄N₂O₂. 0.15 CH₂Cl₂: C, 66.64; H, 5.65; N, 10.98. Found: C, 67.60; H, 5.89; N, 10.98.

### Example 14

### (2E)-3-(4,4-Dimethyl-2-oxo-1,4-dihydro-2H-3,1-benzoxazin-6-yl)acrylic acid

A mixture of methyl(2E)-3-(4,4-dimethyl-2-oxo-1,4-dihydro-2H-3,1-benzoxazin-6-yl)acrylate (0.4 g, 1.53 mmol), prepared in step 3 of Example 2, and 1 N NaOH (2.3 mL, 2.30 mmol) in methanol (5 mL), tetrahydrofuran (5 mL) and water (3 mL) was stirred at room temperature overnight. By LC/MS, starting material remained. The reaction was stirred at 65° C. for 6 hours and then stirred overnight at room temperature. By LC/MS starting material remained. Additional 1 N NaOH (1.53 mL; 1.53 mmol) was added and the reaction was stirred overnight at room temperature. By LC/MS, a small amount of starting material remained. Additional 1 N NaOH (1.53 mL (1.53 mmol) was added and the reaction was stirred over the weekend at room temperature. 2 N HCl (3 mL) was added and the reaction concentrated under reduced pressure. The solid present was collected by filtration and dried under reduced pressure to give a yellow solid (350 mg). Purification of the solid by reverse phase chromatography using acetonitrile-water as the eluent gave the title compound (190 mg, 50%) as a pale yellow solid, mp 260-261° C. (dec), MS (ESI) m/z 248, MS (ESI) m/z 246; Anal. Calcd. for C₁₃H₁₃NO₄. 0.04 C₂H₃N. 0.11 H₂O: C, 62.62; H, 5.36; N, 5.81. Found: C, 62.51; H, 5.44; N, 5.68.

### Example 15

### (2E)-3-(4,4-Dimethyl-2-oxo-1,4-dihydro-2H-3,1-benzoxazin-6-yl)but-2-enoic acid

A mixture of methyl(2E)-3-(4,4-dimethyl-2-oxo-1,4-dihydro-2H-3,1-benzoxazin-6-yl)but-2-enoate (245 mg, 0.89 mmol), prepared in Example 9, 1 N NaOH (1.33 mL, 1.33 mmol), methanol (5 mL), tetrahydrofuran (5 mL) and water (3 mL) was stirred at room temperature overnight. By LC/MS, starting material remained. Additional 1 N NaOH (1.33 mL; 1.33 mmol) was added and the reaction stirred at room temperature overnight. By LC/MS, starting material remained. Again, additional 1 N NaOH (1.33 mL; 1.33 mmol) was added and the reaction was stirred at room temperature overnight. 1 N HCl (4.43 mL) was added and the reaction concentrated under reduced pressure. The solid present was collected by filtration and dried under reduced pressure to give the title compound as an off-white solid, mp 274-275° C. (dec), MS (ESI) m/z 262, MS (ESI) m/z 260; Anal. Calcd. for C₁₄H₁₅NO₄. 0.02 H₂O: C, 64.27; H, 5.79; N, 5.35. Found: C, 64.42; H, 6.11; N, 5.07.

### Example 16

### General Procedure for Examples 16 to 30

A mixture of the appropriate aryl bromide (2.0 mmol), alkylene (2.2 mmol), N,N-dicyclohexylmethylamine (470 μL, 2.2 mmol) and tri-tert-butylphosphine (24 mg, 0.12 mmol) in dioxane (5 mL) was purged with nitrogen. Tris(dibenzylideneacetone)dipalladium (0) (55 mg, 0.06 mmol) was added and the mixture again purged with nitrogen and then stirred at room temperature. The reaction was monitored by LC/MS. If necessary, more catalyst was added. The reaction was diluted with ethyl acetate and filtered through a small amount of silica gel. The silica gel was rinsed with ethyl acetate and then methanol. The filtrates were kept separate and each concentrated under reduced pressure. The residues from the filtrates were analyzed for product by use of LC/MS. The product was purified by the methods described.

### (2E)-3-(2-Oxo-1,2-dihydrospiro[3,1-benzoxazine-4,1′-cyclohexan]-6-yl)but-2-enamide

Step 1: 1-(2-Amino-5-bromo-phenyl)-cyclohexanol

In the same manner as described in step 1 of Example 2, replacing methylmagnesium bromide with pentamethylenebis(magnesium bromide), gave 1-(2-amino-5-bromo-phenyl)-cyclohexanol as a clear oil, MS (ESI) m/z 270.

Step 2: 6-Bromospiro[4H-3,1-benzoxazine-4,1′-cyclohexan]-2(1H)-one

In the same manner as described step 2 of Example 2, replacing 2-(2-amino-5-bromo-phenyl)-propan-2-ol with 1-(2-amino-5-bromo-phenyl)-cyclohexanol, prepared in the previous step, gave 6-bromospiro[4H-3,1-benzoxazine-4,1′-cyclohexan]-2(1H)-one as a yellow solid, mp 208-210° C., MS (APCI) m/z 296.

Step 3: (2E)-3-(2-Oxo-1,2-dihydrospiro[3,1-benzoxazine-4,1′-cyclohexan]-6-yl)but-2-enamide

6-Bromospiro[4H-3,1-benzoxazine-4,1′-cyclohexan]-2(1H)-one (592 mg, 2.0 mmol), prepared in the previous step, and (E)-but-2-enamide (187 mg, 2.2 mmol) were reacted according to the General Procedure described above. By LC/MS, the methanol filtrate contained the product. The methanol filtrate was concentrated under reduced pressure to remove the solvent. The residue was taken up in ethyl acetate. Upon standing some solid precipitated. The solid was collected by filtration and dried under reduced pressure. By nuclear magnetic resonance (NMR) analysis, the solid contained ethyl acetate. The solid was taken up in methanol-methylene chloride and concentrated under reduced pressure. It was then taken up in methylene chloride and again concentrated under reduced pressure. This process was repeated two additional times and then the solid was dried under reduced pressure to give the title compound (50 mg, 8%) as a white solid, mp 204-207° C. (dec), MS m/z 301, MS m/z 299.

### Example 17

### (2E)-3-(2-oxo-1,2-dihydrospiro[3,1-benzoxazine-4,1′-cyclohexan]-6-yl)but-2-enenitrile

A mixture of (2E)-3-(2-oxo-1,2-dihydrospiro[3,1-benzoxazine-4,1′-cyclohexan]-6-yl)but-2-enamide (300 mg, 1 mmol), prepared in step 3 of Example 16, and thionyl chloride (510 μL, 7 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave the title compound (204 mg, 72%) as a pale yellow solid, mp 208-210° C. (dec), MS (ES) m/z 281.1; Anal. Calcd. for C₁₇H₁₈N₂O₂. 0.09 CH₂Cl₂: C, 70.79; H, 6.32; N, 9.66. Found: C, 70.48; H, 6.31; N, 9.48.

### Example 18

### (2E)-3-(2-Oxo-1,2-dihydrospiro[3,1-benzoxazine-4,1′-cyclohexan]-6-yl)pent-2-enamide

The reaction was run using the General Procedure described in Example 16 and replacing (E)-but-2-enamide with (E)-pent-2-enamide (218 mg, 2.2 mmol). Purification of the crude product on a Horizon™ Flash 25+™ M silicon column (Biotage) using a gradient of 0.5% methanol:methylene chloride to 10% methanol:methylene chloride gave an oily solid (550 mg). By LC/MS, the material was not pure. Recrystallization of the oily solid from methanol-acetonitrile gave the title compound (70 mg, 11%) as a white solid, mp 235-238° C. (dec), MS (ES) m/z 314.2.

### Example 19

### (2E)-3-(2-Oxo-1,2-dihydrospiro[3,1-benzoxazine-4,1′-cyclohexan]-6-yl)pent-2-enenitrile

A mixture of (2E)-3-(2-oxo-1,2-dihydrospiro[3,1-benzoxazine-4,1′-cyclohexan]-6-yl)pent-2-enamide (250 mg, 0.795 mmol), prepared in Example 18, and thionyl chloride (410 μL, 5.56 mmol) in dioxane (20 mL) was stirred under nitrogen at 75° C. overnight. The reaction was concentrated under reduced pressure. The residue was taken up in metlhylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave 70 mg of a material which by LC/MS was not pure. Additional purification by reverse phase chromatography using a Luna® C8 250×20 mm column using a gradient of 50/50 to 30/70 water acetonitrile over 15 minutes gave the title compound (35 mg, 15%) as a white solid, mp 173-174° C., MS (ESI) m/z 297, MS (ESI) m/z 295; Anal. Calcd. for 1.00 C₁₈H₂₀N₂O₂+0.04 CH₂Cl₂: C, 72.95; H, 6.80; N, 9.45. Found: C, 72.22; H, 6.69; N, 9.11.

### Example 20

### (2E)-3-(2-Oxo-1,2-dihydrospiro[3,1-benzoxazine-4,1′-cyclohexan]-6-yl)hex-2-enenitrile

Step 1: (2E)-3-(2-Oxo-1,2-dihydrospiro[3,1-benzoxazine-4,1′-cyclohexan]-6-yl)hex-2-enamide

The reaction was run using the General Procedure described in Example 16 and replacing (E)-but-2-enamide with (E)-hex-2-enamide (249 mg, 2.2 mmol). Purification of the crude product on a Horizon™ Flash 25+™ M silicon column (Biotage) using a gradient of 0.5% methanol:methylene chloride to 10% methanol:methylene chloride gave (2E)-3-(2-oxo-1,2-dihydrospiro[3,1-benzoxazine-4,1′-cyclohexan]-6-yl)hex-2-enamide (216 mg) as a yellow solid. The material was used in step 2 without additional purification.

Step 2: (2E)-3-(2-Oxo-1,2-dihydrospiro[3,1-benzoxazine-4,1′-cyclohexan]-6-yl)hex-2-enenitrile

A mixture of (2E)-3-(2-oxo-1,2-dihydrospiro[3,1-benzoxazine-4,1′-cyclohexan]-6-yl)hex-2-enamide (200 mg, 0.609 mmol), prepared in the previous step, and thionyl chloride (310 μL, 4.26 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave a material (40 mg) which by LC/MS was not pure. Additional purification by reverse phase chromatography using an Xterra™ RP18 column, 3.5 μ, 150×4.6 mm column and a gradient of 85/15-5/95 (NH₄OAc buffer; pH=3.5/acetonitrile-methanol) over 10 minutes and holding for 4 minutes gave the title compound as a pale yellow solid, MS (ES) m/z 309.2.

### Example 21

### (2E)-4-Methyl-3-(2-oxo-1,2-dihydrospiro[3,1-benzoxazine-4,1′-cyclohexan]-6-yl)pent-2-enenitrile

Step 1: (2E)-4-Methyl-3-(2-oxo-1,2-dihydrospiro[3,1-benzoxazine-4,1′-cyclohexan]-6-yl)pent-2-eneamide

The reaction was run using the General Procedure described in Example 16 and replacing (E)-but-2-enamide with (E)-4-methylpent-2-enamide (249 mg, 2.2 mmol). Purification of the crude product on a Horizon™ Flash 25+™ M silicon column (Biotage) using a gradient of 0.5% methanol:methylene chloride to 10% methanol:methylene chloride gave (2E)-4-methyl-3-(2-oxo-1,2-dihydrospiro[3,1-benzoxazine-4,1′-cyclohexan]-6-yl)pent-2-eneamide (92 mg). The material was used in step 2 without additional purification.

Step 2: (2E)-4-Methyl-3-(2-oxo-1,2-dihydrospiro[3,1-benzoxazine-4,1′-cyclohexan]-6-yl)pent-2-enenitrile

A mixture of (2E)-4-methyl-3-(2-oxo-1,2-dihydrospiro[3,1-benzoxazine-4,1′-cyclohexan]-6-yl)pent-2-eneamide (100 mg, 0.304 mmol), prepared in the previous step, and thionyl chloride (160 μL, 2.13 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave 60 mg of a material which by LC/MS was not pure. Additional purification by reverse phase chromatography using an Xterra™ RP18, 3.5 μ, 150×4.6 mm column using a gradient of 85/15-5/95 (NH₄OAc buffer; pH=3.5/acetonitrile-methanol) over 10 minutes and holding for 4 minutes gave the title compound as a white solid, mp 201-203° C. (dec), MS (ES) m/z 309.2.

### Example 22

### (2E)-3-(4,4-Diethyl-2-oxo-1,4-dihydro-2H-3,1-benzoxazin-6-yl)but-2-enenitrile

Step 1: 3-(2-Amino-5-bromophenyl)pentan-3-ol

In the same manner as described in step 1 of Example 2, replacing 2-amino-5-bromobenzoic acid with methyl 2-amino-5-bromobenzoate and methylmagnesium bromide with ethyl magnesium bromide, 3-(2-amino-5-bromophenyl)pentan-3-ol was isolated a tan solid, mp 64-66° C., MS (ESI) m/z 258; Anal. Calcd. for C₁₁H₁₆BrNO: C, 51.18; H, 6.25; N, 5.43. Found: C, 51.22; H, 6.22; N, 5.54.

Step 2: 6-Bromo-4,4-diethyl-1,4-dihydro-2H-3,1-benzoxazin-2-one

In the same manner as described step 2 of Example 2, replacing 2-(2-amino-5-bromo-phenyl)-propan-2-ol with 3-(2-amino-5-bromophenyl)pentan-3-ol, prepared in the previous step, 6-bromo-4,4-diethyl-1,4-dihydro-2H-3,1-benzoxazin-2-one was isolated as a white solid, mp 150-152° C., MS (ESI) m/z 284, MS (ESI) m/z 282; Anal. Calcd. for C₁₂H₁₄BrNO₂: C, 50.72; H, 4.97; N, 4.93. Found: C, 50.47; H, 4.80; N,4.74.

Step 3: (2E)-3-(4,4-Diethyl-2-oxo-1,4-dihydro-2H-3,1-benzoxazin-6-yl)but-2-eneamide

The reaction was run using the General Procedure described before Example 16 and replacing 6-bromospiro[4H-3,1-benzoxazine-4,1′-cyclohexan]-2(1H)-one with 6-bromo-4,4-diethyl-1,4-dihydro-2H-3,1-benzoxazin-2-one (568 mg, 2.0 mmol) prepared in the previous step. Purification of the crude product on a Horizon™ Flash 25+™ M silicon column (Biotage) using a gradient of 0.5% methanol:methylene chloride to 10% methanol:methylene chloride gave (2E)-3-(4,4-diethyl-2-oxo-1,4-dihydro-2H-3,1-benzoxazin-6-yl)but-2-eneamide (500 mg) as a white foam. The material was used in step 4 without additional purification.

Step 4: (2E)-3-(4,4-Diethyl-2-oxo-1,4-dihydro-2H-3,1-benzoxazin-6-yl)but-2-enenitrile

A mixture of (2E)-3-(4,4-diethyl-2-oxo-1,4-dihydro-2H-3,1-benzoxazin-6-yl)but-2-eneamide (250 mg, 0.867 mmol), prepared in the previous step, and thionyl chloride (440 μL, 6.069 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave a material (110 mg) which by LC/MS was not pure. Additional purification by reverse phase chromatography using a Luna® C18 250×50 mm column and 45% acetonitrile/55% water as the eluent gave the title compound (100 mg, 43%) as a white solid, mp 159-160° C., MS (ESI) m/z 271, MS (ESI) m/z 269; Anal. Calcd. for C₁₆H₁₈N₂O₂. 0.05 CH₂Cl₂: C, 70.21; H, 6.64; N, 10.20. Found: C, 70.29; H, 6.88; N, 10.1.

### Example 23

### (2E)-3-(4,4-Diethyl-2-oxo-1,4-dihydro-2H-3,1-benzoxazin-6-yl)pent-2-enenitrile

Step 1: (2E)-3-(4,4-Diethyl-2-oxo-1,4-dihydro-2H-3,1-benzoxazin-6-yl)pent-2-eneamide

The reaction was run using the General Procedure described in Example 16 and replacing 6-bromospiro[4H-3,1-benzoxazine-4,1′-cyclohexan]-2(1H)-one with 6-bromo-4,4-diethyl-1,4-dihydro-2H-3,1-benzoxazin-2-one (568 mg, 2.0 mmol), prepared in step 2 of Example 22 and (E)-but-2-enamide with (E)-pent-2-enamide (218 mg, 2.2 mmol). Purification of the crude product on a Horizon™ Flash 25+™ M silicon column (Biotage) using a gradient of 0.5% methanol:methylene chloride to 10% methanol:methylene chloride gave (2E)-3-(4,4-diethyl-2-oxo-1,4-dihydro-2H-3,1-benzoxazin-6-yl)pent-2-eneamide (363 mg) as a yellow foam. The material was used in step 2 without additional purification.

Step 2: (2E)-3-(4,4-Diethyl-2-oxo-1,4-dihydro-2H-3,1-benzoxazin-6-yl)pent-2-enenitrile

A mixture of (2E)-3-(4,4-diethyl-2-oxo-1,4-dihydro-2H-3,1-benzoxazin-6-yl)pent-2-eneamide (300 mg, 0.992 mmol), prepared in the previous step, and thionyl chloride (510 μL, 6.945 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave a material (110 mg) which by LC/MS was not pure. Additional purification by reverse phase chromatography using a Luna® C18 250×50 mm column and 40% acetonitrile/60% water as the eluent gave the title compound (50 mg, 18%) as a pale yellow solid, mp 135-136° C., MS (ES) m/z 283.1.

### Example 24

### (2E)-3-(4,4-Diethyl-2-oxo-1,4-dihydro-2H-3,1-benzoxazin-6-yl)hex-2-enamide

The reaction was run using the General Procedure described in Example 16 and replacing 6-bromospiro[4H-3,1-benzoxazine-4,1′-cyclohexan]-2(1H)-one with 6-bromo-4,4-diethyl-1,4-dihydro-2H-3,1-benzoxazin-2-one (568 mg, 2.0 mmol), prepared in step 2 of Example 22 and (E)-but-2-enamide with (E)-hex-2-enamide (249 mg, 2.2 mmol). Purification of the crude product from the methanol rinse on a Horizon™ Flash 25+™ M silicon column (Biotage) using a gradient of 0.5% methanol:methylene chloride to 10% methanol:methylene chloride gave the title compound (233 mg, 37%) of a yellow solid. Additional material (280 mg) was isolated from the ethyl acetate rinse. Purification of this material on a Horizon™ Flash 25+™ M silicon column (Biotage) using a gradient of 0.5% methanol:methylene chloride to 10% methanol:methylene chloride gave a material which by LC/MS was not pure. Additional purification by reverse phase chromatography using a Luna® C18 250×50 mm column and 40% acetonitrile/60% water as the eluent gave additional title compound (102 mg; 16%) as a white solid, mp 212-213° C., MS (ESI) m/z 317, MS (ESI) m/z 315.

### Example 25

### (2E)-3-(4,4-Diethyl-2-oxo-1,4-dihydro-2H-3,1-benzoxazin-6-yl)hex-2-enenitrile

A mixture of (2E)-3-(4,4-diethyl-2-oxo-1,4-dihydro-2H-3,1-benzoxazin-6-yl)hex-2-enamide (80 mg, 0.253 mmol), prepared in Example 24, and thionyl chloride (130 μL, 1.77 mmol) in dioxane (15 mL) was stirred under nitrogen at 70° C. for 6 hours. After standing overnight at room temperature, the reaction was concentrated under reduced pressure. Purification of the crude product on a Horizon™ Flash 25+™ M silicon column (Biotage) using of 25% ethyl acetate:methylene chloride as the eluent gave title compound (60 mg, 79%) as an off-white solid, mp 121-124° C., MS m/z 299, MS m/z 297.

### Example 26

### (2E)-3-(4,4-Diethyl-2-oxo-1,4-dihydro-2H-3,1-benzoxazin-6-yl)hex-2-enal

(E)-Hex-2-enal (450 μL, 3.88 mmol) and N,N-dicyclohexylmethylamine (832 μL 3.88 mmol) were added to a suspension of 6-bromo-4,4-diethyl-1,4-dihydro-2H-3,1-benzoxazin-2-one (1.0024 g, 3.53 mmol), prepared in step 2 of Example 22, in anhydrous dioxane (10 mL) and the resulting solution put under nitrogen. Tri-tert-butylphosphine (638 μL, 10% by weight in hexane; 0.212 mmol) was added and the solution purged with nitrogen. Tris(dibenzylideneacetone)dipalladium (0) (103.7 mg, 0.113 mmol) was added and the mixture again purged with nitrogen. The reaction was stirred under nitrogen at 100° C. for 6 hours, stood overnight at room temperature and then concentrated under reduced pressure. Purification of the residue on silica gel (500 g; 230-400 mesh) using 3:2 hexane:ethyl acetate as the eluent gave 611.7 mg of a yellow solid. NMR analysis of the solid indicated that it was a mixture of components. Additional purification by reverse phase chromatography using a Luna® C18 250×50 mm column and a gradient of acetonitrile/water gave the title compound (343.1 mg, 32%) as a white solid, mp 173-179° C., MS (ESI) m/z 302.

### Example 27

### (2E)-3-(4,4-Diethyl-2-oxo-1,4-dihydro-2H-3,1-benzoxazin-6-yl)-4-methylpent-2-enenitrile

Step 1: (2E)-3-(4,4-Diethyl-2-oxo-1,4-dihydro-2H-3,1-benzoxazin-6-yl)-4-methylpent-2-enamide

The reaction was run using the General Procedure described in Example 16 and replacing 6-bromospiro[4H-3,1-benzoxazine-4,1′-cyclohexan]-2(1H)-one with 6-bromo-4,4-diethyl-1,4-dihydro-2H-3,1-benzoxazin-2-one (568 mg, 2.0 mmol), prepared in step 2 of Example 22 and (E)-but-2-enamide with (E)-4-methylpent-2-enamide (249 mg, 2.2 mmol). Purification of the crude product on a Horizon™ Flash 25+™ M silicon column (Biotage) using a gradient of 0.5% methanol:methylene chloride to 10% methanol:methylene chloride gave (2E)-3-(4,4-diethyl-2-oxo-1,4-dihydro-2H-3,1-benzoxazin-6-yl)-4-methylpent-2-enamide (231 mg) as a yellow foam. The material was used in step 2 without additional purification.

Step 2: (2E)-3-(4,4-Diethyl-2-oxo-1,4-dihydro-2H-3,1-benzoxazin-6-yl)-4-methylpent-2-enenitrile

A mixture of (2E)-3-(4,4-diethyl-2-oxo-1,4-dihydro-2H-3,1-benzoxazin-6-yl)-4-methylpent-2-enamide (230 mg, 0.727 mmol), prepared in the previous step, and thionyl chloride (370 μL, 5.08 mmol) in dioxane (20 mL) was stirred under nitrogen at 75° C. overnight. The reaction was concentrated under reduced pressure. The residue was taken up in methylene chloride and concentrated under reduced pressure. This process was repeated three additional times. Purification of the crude product on a Horizon™ Flash 25+™ M silicon column (Biotage) using a gradient of 20% ethyl acetate:hexane to 50% ethyl acetate:hexane gave the title compound (45 mg, 21%) as atan solid, mp 138-140° C., MS (ES) m/z 297.1.

### Example 28

### (2E)-3-(4,4-Dimethyl-2-oxo-1,4-dihydro-2H-3,1-benzoxazin-6-yl)pent-2-enenitrile

Step 1: (2E)-3-(4,4-Dimethyl-2-oxo-1,4-dihydro-2H-3,1-benzoxazin-6-yl)pent-2-enamide

The reaction was run using the General Procedure described in Example 16 and replacing 6-bromospiro[4H-3,1-benzoxazine-4,1′-cyclohexan]-2(1H)-one with 6-bromo-4,4-dimethyl-1,4-dihydro-2H-3,1-benzoxazin-2-one (512 mg, 2.0 mmol), prepared in step 2 of Example 2, and (E)-but-2-enamide with (E)-pent-2-enamide (218 mg, 2.2 mmol). Purification of the crude product on a Horizon™ Flash 25+™ M silicon column (Biotage) using a gradient of 0.5% methanol:methylene chloride to 10% methanol:methylene chloride gave a mushy solid (175 mg) which by LC/MS was not pure. Additional purification by reverse phase chromatography using a Luna® C18 250×50 mm column and a gradient of 20-43% acetonitrile/water over 15 minutes gave (2E)-3-(4,4-dimethyl-2-oxo-1,4-dihydro-2H-3,1-benzoxazin-6-yl)pent-2-enamide (10 mg). The material was used in step 2 without additional purification.

Step 2: (2E)-3-(4,4-Dimethyl-2-oxo-1,4-dihydro-2H-3,1-benzoxazin-6-yl)pent-2-enenitrile

A mixture of (2E)-3-(4,4-dimethyl-2-oxo-1,4-dihydro-2H-3,1-benzoxazin-6-yl)pent-2-enamide (10 mg, 0.036 mmol), prepared in the previous step, and thionyl chloride (100 μL, 1.371 mmol) in dioxane (10 mL) was stirred under nitrogen at 75° C. for 6 hours. The mixture was concentrated under reduced pressure to remove the volatiles. Purification of the residue on a small amount of silica gel using 20% ethyl acetate-methylene chloride as the eluent gave the title compound (6 mg, 65%) as a pale yellow solid, MS (ESI) m/z 257, MS (ESI) m/z 255.

### Example 29

### (2E)-3-(4,4-Dimethyl-2-oxo-1,4-dihydro-2H-3,1-benzoxazin-6-yl)hex-2-enenitrile

Step 1: (2E)-3-(4,4-Dimethyl-2-oxo-1,4-dihydro-2H-3,1-benzoxazin-6-yl)hex-2-enamide

The reaction was run using the General Procedure described in Example 16 and replacing 6-bromospiro[4H-3,1-benzoxazine-4,1′-cyclohexan]-2(1H)-one with 6-bromo-4,4-dimethyl-1,4-dihydro-2H-3,1-benzoxazin-2-one (512 mg, 2.0 mmol), prepared in step 2 of Example 2 and (E)-but-2-enamide with (E)-hex-2-enamide (249 mg, 2.2 mmol). Purification of the crude product on a Horizon™ Flash 25+™ M silicon column (Biotage) using a gradient of 0.5% methanol:methylene chloride to 10% methanol:methylene chloride gave a residue (235 mg) which by LC/MS was not pure. Additional purification by reverse phase chromatography using a Luna™ C18 250×50 mm column and a gradient of 20-43% acetonitrile/water over 17 minutes gave (2E)-3-(4,4-dimethyl-2-oxo-1,4-dihydro-2H-3,1-benzoxazin-6-yl)hex-2-enamide (11 mg). The material was used in step 2 without additional purification.

Step 2: (2E)-3-(4,4-Dimethyl-2-oxo-1,4-dihydro-2H-3,1-benzoxazin-6-yl)hex-2-enenitrile

A mixture of (2E)-3-(4,4-dimethyl-2-oxo-1,4-dihydro-2H-3,1-benzoxazin-6-yl)hex-2-enamide (11 mg, 0.038 mmol), prepared in the previous step, and thionyl chloride (50 μL, 0.685 mmol) in dioxane (10 mL) was stirred under nitrogen at 70° C. for 6 hours. The mixture was concentrated under reduced pressure to remove the volatiles. Purification of the residue on a small amount of silica gel using 25% ethyl acetate-methylene chloride as the eluent gave the title compound (4 mg, 39%) as an off-white solid, MS (ESI) m/z 271, MS (ESI) m/z 269.

### Example 30

### (2E)-3-(4,4-Dimethyl-2-oxo-1,4-dihydro-2H-3,1-benzoxazin-6-yl)-4-methylpent-2-enenitrile

Step 1: (2E)-3-(4,4-Dimethyl-2-oxo-1,4-dihydro-2H-3,1-benzoxazin-6-yl)-4-methylpent-2-enamide

The reaction was run using the General Procedure described in Example 16 and replacing 6-bromospiro[4H-3,1-benzoxazine-4,1′-cyclohexan]-2(1H)-one with 6-bromo-4,4-dimethyl-1,4-dihydro-2H-3,1-benzoxazin-2-one (512 mg, 2.0 mmol), prepared in step 2 of Example 2 and (E)-but-2-enamide with (E)-4-methylpent-2-enamide (249 mg, 2.2 mmol). Purification of the crude product on a Horizon™ Flash 25+™ M silicon column (Biotage) and a gradient of 0.5% methanol:methylene chloride to 10% methanol:methylene chloride gave a residue (315 mg) which by LC/MS was not pure. Additional purification by reverse phase chromatography using a Luna® C18 250×50 mm column and a gradient of 20-43% acetonitrile/water over 15 minutes gave (2E)-3-(4,4-dimethyl-2-oxo-1,4-dihydro-2H-3,1-benzoxazin-6-yl)-4-methylpent-2-enamide (17 mg). The material was used in step 2 without additional purification.

Step 2: (2E)-3-(4,4-dimethyl-2-oxo-1,4-dihydro-2H-3,1-benzoxazin-6-yl)-4-methylpent-2-enenitrile

A mixture of (2E)-3-(4,4-dimethyl-2-oxo-1,4-dihydro-2H-1-3,1-benzoxazin-6-yl)-4-methylpent-2-enamide (17 mg, 0.059 mmol), prepared in the previous step, and thionyl chloride (100 μL, 1.371 mmol) in dioxane (10 mL) was stirred under nitrogen at 75° C. for 6 hours. The mixture was concentrated under reduced pressure to remove the volatiles. Purification of the residue on a small amount of silica gel using 20% ethyl acetate-methylene chloride as the eluent gave the title compound (10 mg, 63%) as a white solid, MS (ESI) m/z 271, MS (ESI) m/z 269.

### Example 31

### Effects of progestins and antiprogestins on alkaline phosphatase activity in T47D cells

The purpose of this example is to identify progestins or antiprogestins by determining a compound's effect on alkaline phosphatase activity in T47D cells.

### Materials and Methods:

A. Reagents:
Culture medium: Dulbecco's Modified Eagle Medium (DMEM):F12 (1:1) (GIBCO, BRL) supplemented with 5% (v/v) charcoal stripped fetal bovine serum (not heat-inactivated), 100 U/mL penicillin, 100 μg/mL streptomycin, and 2 mM of the GlutaMax® reagent (GIBCO, BRL).
Alkaline phosphatase assay buffer:
   I. 0.1M Tris-HCl, pH 9.8, containing 0.2% Triton® X-100 reagent
   II. 0.1M Tris-HCl, pH 9.8, containing 4 mM p-nitrophenyl phosphate (Sigma).

B. Cell Culture and Treatment:

Frozen T47D cells are thawed in a 37° C. water bath and diluted to 280,000 cells/mL in culture medium. To each well in a 96-well plate (Falcon, Becton Dickinson Labware), was added 180 μL of diluted cell suspension. Twenty μL of reference or test compounds diluted in the culture medium is then added to each well. When testing for progestin antagonist activity, reference antiprogestins or test compounds are added in the presence of 1 nM progesterone. The cells are incubated at 37° C. in a 5% CO₂/humidified atmosphere for 24 hours. For high throughput screening, one concentration of each compound will be tested at 0.3 μg/mL. Based on an average molecular weight of 300 g/mol for the compounds in the library, the concentration is approximately 1 μM. Compounds active in this assay can be tested in dose response assays to determine EC₅₀ and IC₅₀.

C. Alkaline Phosphatase Enzyme Assay:
At the end of treatment, the medium is removed from the plate. Fifty μL of assay buffer I is added to each well. The plates are shaken in a titer plate shaker for 15 minutes. Then 150 μL of assay buffer II is added to each well. Optical density measurements are taken at 5 minute intervals for 30 minutes at a test wavelength of 405 nM.

D. Analysis of Results: Analysis of Dose-Response Data
For reference and test compounds, a dose response curve is generated for dose (X-axis) vs. the rate of enzyme reaction (slope) (Y-axis). Square root-transformed data are used for analysis of variance and nonlinear dose response curve fitting for both agonist and antagonist modes. Huber weighting is used to down-weight the effects of outliers. EC₅₀ or IC₅₀ values are calculated from the retransformed values. JMP® software (SAS Institute, Inc.) is used for both one-way analysis of variance and non-linear dose response analysis in both single dose and dose response studies.

E. Reference Compounds
Progesterone and trimegestone are reference progestins and RU486 is the reference antiprogestin. All reference compounds are run in full dose response curves and the EC₅₀ and IC₅₀ values are calculated.

### Example 32

### Progesterone receptor whole cell competition binding assay using T47D cells

The purpose of this example is to evaluate the progesterone receptor (PR) binding activity of progestins or antiprogestins in live, intact (whole) cells, using the human breast carcinoma T47D cell line and ³H-progesterone as the labeled ligand.

### Materials and Methods:

A. Reagents:
Culture medium:
   (i) 5% RC which is phenol red free Dulbecco's Modified Eagles Medium (DMEM):F12 (1:1) (GIBCO, BRL) supplemented with 5% (v/v) charcoal stripped fetal bovine serum (not heat-inactivated), 100 U/mL penicillin, 100 μg/mL streptomycin, and 2 mM of the GlutaMax™ reagent (GIBCO, BRL)
   (ii) 10% RC is 5% RC supplemented with 10% (v/v) FBS
   ³H-Progesterone: Perkin Elmer Life Science, cat# NET-381 (typically around 102 Ci/mmol)
   Liquid Scintillation Cocktail: Beckman Coulter, the Ready-Safe™ reagent; cat# 141349
   Tissue Culture Plates: 96 well, clear bottom, white, plates (VWR Part # 29443-150 or Perkin Elmer Part # 3983498)

B. T47D Cell Culture:
T47D cells are maintained in 10% RC media at 37° C. in a 5% CO₂/humidified atmosphere and need to be split twice weekly for proper response. Cells are plated in 10% RC the day before binding assay at 50,000 cells per well in the white, clear bottom plates purchased through VWR or Perkin Elmer.

C. Binding Assay:

Cells plated the day prior to the assay in white clear bottom plates are used. A master compound plate is set up containing control and test compounds at 20X final desired concentration for the competition binding. A typical dose range of 20X concentrations are (in nM); 200,000; 20,000; 6000; 2000; 600; 200; 20; and 2. Final concentrations are then (in nM); 10,000; 1000; 300; 100; 30; 10; 1; 0.1. Control compounds are typically run 10-fold lower than this and include a 0, or vehicle, control well. A stock of 60 nM ³H-progesterone (20X) is also prepared at a volume needed of 10 μL per well.

Media on cells are replaced with 180 μL of 5% RC. Ten microliters (10 μL) of 60 nM ³H-progesterone (for final concentration of 3 nM) is added immediately followed by 10 μL of 20X test or control compounds. Compounds are incubated for 3 hours at 37° C. A time course study found no difference between 2 and 4 hours incubation.

Following incubation, media is carefully removed and cells are washed 3X with 200 μL 5% RC each wash. Fifty microliters of liquid scintillation cocktail is added and the plates are shaken vigorously for a minimum of 15 minutes. Plates are read on the Wallac Microbeta® 1450 plate reader.

D. Analysis of Results:
Square root-transformed data are used for analysis of variance and calculation of IC₅₀. SAS software (SAS Institute, Inc.) is used for all the statistical analysis.

E. Reference Compounds:
Progesterone is used as a reference progestin and RU486 as a reference antiprogestin.

| | T47D Alkaline Phosphatase | | PR T47D Whole |
|---|---|---|---|
| | Activity IC₅₀ (nM) | | Cell Binding IC₅₀ |
| Example | or % inhibition | | (nM) |
| 1 | 3000 | | |
| 2 | 3000 | | |
| 3 | 3000 | | |
| 4 | 50000 | | |
| 5 | 50000 | | |
| 6 | 80.3 | | 82.5 |
| 7 | 50000 | | |
| 8 | 3000 | | |
| 9 | 61.8 | | 84.8 |
| 10 | 3000 | | |
| 11 | 3000 | | |
| 12 | 25% at 3000 | nM | 1395 |
| 13 | 38.2 | | 48.1 |
| 14 | 0% at 3000 | nM | |
| 15 | 0% at 3000 | nM | |
| 16 | 0% at 3000 | nM | |
| 17 | 60% at 300 | nM | |
| 18 | 22% at 3000 | nM | |
| 19 | 60% at 30 | nM | |
| 20 | 55.7 | | 46.5 |
| 21 | 60% at 300 | nM | |
| 22 | 49.6 | | 150 |
| 23 | 29.2 | | 52.8 |
| 24 | 65% at 3000 | nM | |
| 25 | 16.1 | | 49.2 |
| 26 | 100% at 3000 | nM | |
| 27 | 208.5 | | 1421 |
| 28 | 70% at 30 | nM | 89.8 |
| 29 | 84% at 300 | nM | 104 |
| 30 | 84.6 | | 777 |

All publications cited in this specification are incorporated herein by reference herein. While the invention has been described with reference to a particularly preferred embodiment, it will be appreciated that modifications can be made without departing from the spirit of the invention. Such modifications are intended to fall within the scope of the appended claims.

What is claimed is:

1. A compound of formula I, or a pharmaceutically acceptable salt thereof: 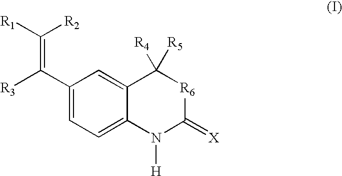
wherein:
R₁ is selected from the group consisting of H, —CO₂—C₁—C₃ alkyl, —CO₂H, —CR₇O, —CONR₈R₉, —CN, and isoxazole;
R₂ is selected from the group consisting of H, —CO₂—C₁—C₃-alkyl, —CO₂H, —CR₇O, —CONR₈R₉, —CN, C₁-C₆ alkyl, aryl, and heteroaryl;
with the proviso that both R₁ and R₂ are not H;
R₃ is H, C₁-C₆ alkyl, aryl, or heteroaryl;
or R₁ and R₃ together form a 6-membered lactone, wherein a 2-carbon bridge is formed between the CO₂—C₁—C₃ alkyl of R₁ and an alkyl group of R₃;
R₄ and R₅ are independently C₁-C₃ alkyl or together with the carbon to which they are attached form a saturated 3 to 6-membered carbon-based ring;
R₆ is O or CR₁₀R₁₁;
R₇, R₈ and R₉ are independently selected from the group consisting of H and C₁-C₃ alkyl;
R₁₀ and R₁₁ are independently H or C₁-C₃ alkyl; or
R₁₀ and R₁₁ together with the carbon to which they are attached form a saturated 3 to 6-membered carbon-based ring; and
X is O or S.

2. The compound according to claim 1, wherein R₁ has the E stereochemistry relative to the aryl ring containing the R₄ and R₅ substituents.

3. The compound according to claim 1, wherein R₁ is CN.

4. The compound according to claim 1, wherein R₄ and R₅ are methyl or ethyl.

5. The compound according to claim 1, wherein X is O.

6. The compound according to claim 1, wherein said 6-membered lactone is of the structure:

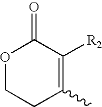

7. The compound according to claim 1, selected from the group consisting of:
Methyl-3-(4,4-dimethyl-2-oxo-1,2,3,4-tetrahydroquinolin-6-yl)acrylate;
Methyl-3-(4,4-dimethyl-2-oxo-1,4-dihydro-2H-3,1-benzoxazin-6-yl)acrylate;
6-[2-Isoxazol-5-ylvinyl]-4,4-dimethyl-1,4-dihydro-2H-3,1-benzoxazin-2-one;
3-(4,4-Dimethyl-2-oxo-1,4-dihydro-2H-3,1-benzoxazin-6-yl)acrylamide;
3-(4,4-Dimethyl-2-oxo-1,4-dihydro-2H-3,1-benzoxazin-6-yl)but-2-enamide;
3-(4,4-Dimethyl-2-oxo-1,4-dihydro-2H-3,1-benzoxazin-6-yl)-3-phenylacrylonitrile;
3-(4,4-Dimethyl-2-oxo-1,4-dihydro-2H-3,1-benzoxazin-6-yl)-3-phenylacrylamide;
3-(4,4-Dimethyl-2-oxo-1,4-dihydro-2H-3,1-benzoxazin-6-yl)acrylonitrile;
Methyl-3-(4,4-dimethyl-2-oxo-1,4-dihydro-2H-3,1-benzoxazin-6-yl)but-2-enoate;
4,4-Dimethyl-6-(6-oxo-3,6-dihydro-2H-pyran-4-yl)-1,4-dihydro-2H-3,1-benzoxazin-2-one;
Methyl-3-(4,4-dimethyl-2-oxo-1,4-dihydro-2H-3,1-benzoxazin-6-yl)-3-phenylacrylate;
3-(4,4-Dimethyl-2-oxo-1,4-dihydro-2H-3,1-benzoxazin-6-yl)but-2-enenitrile;
3-(4,4-Dimethyl-2-oxo-1,4-dihydro-2H-3,1-benzoxazin-6-yl)acrylic acid;
3-(4,4-Dimethyl-2-oxo-1,4-dihydro-2H-3,1-benzoxazin-6-yl)but-2-enoic acid;
3-(2-Oxo-1,2-dihydrospiro[3,1-benzoxazine-4,1′-cyclohexan]-6-yl)but-2-enamide;
3-(2-oxo-1,2-dihydrospiro[3,1-benzoxazine-4,1′-cyclohexan]-6-yl)but-2-enenitrile;
3-(2-Oxo-1,2-dihydrospiro[3,1-benzoxazine-4,1′-cyclohexan]-6-yl)pent-2-enamide;
3-(2-Oxo-1,2-dihydrospiro[3,1-benzoxazine-4,1′-cyclohexan]-6-yl)pent-2-enenitrile;
3-(2-Oxo-1,2-dihydrospiro[3,1-benzoxazine-4,1′-cyclohexan]-6-yl)hex-2-enenitrile;
4-methyl-3-(2-oxo-1,2-dihydrospiro[3,1-benzoxazine-4,1′-cyclohexan]-6-yl)pent-2-enenitrile;
3-(4,4-Diethyl-2-oxo-1,4-dihydro-2H-3,1-benzoxazin-6-yl)but-2-enenitrile;
3-(4,4-Diethyl-2-oxo-1,4-dihydro-2H-3,1-benzoxazin-6-yl)pent-2-enenitrile;
3-(4,4-Diethyl-2-oxo-1,4-dihydro-2H-3,1-benzoxazin-6-yl)hex-2-enamide;
3-(4,4-Diethyl-2-oxo-1,4-dihydro-2H-3,1-benzoxazin-6-yl)hex-2-enenitrile;
3-(4,4-Diethyl-2-oxo-1,4-dihydro-2H-3,1-benzoxazin-6-yl)hex-2-enal;
3-(4,4-Diethyl-2-oxo-1,4-dihydro-2H-3,1-benzoxazin-6-yl)-4-methylpent-2-enenitrile;
3-(4,4-Dimethyl-2-oxo-1,4-dihydro-2H-3,1-benzoxazin-6-yl)pent-2-enenitrile;
3-(4,4-Dimethyl-2-oxo-1,4-dihydro-2H-3,1-benzoxazin-6-yl)hex-2-enenitrile; and
3-(4,4-Dimethyl-2-oxo-1,4-dihydro-2H-3,1-benzoxazin-6-yl)-4-methylpent-2-enenitrile, and
pharmaceutically acceptable salts thereof.

8. The compound according to claim 6, selected from the group consisting of:
Methyl(2E)-3-(4,4-dimethyl-2-oxo-1,2,3,4-tetrahydroquinolin-6-yl)acrylate;
Methyl(2E)-3-(4,4-dimethyl-2-oxo-1,4-dihydro-2H-3,1-benzoxazin-6-yl)acrylate;
6-[(E)-2-Isoxazol-5-ylvinyl]-4,4-dimethyl-1,4-dihydro-2H-3,1-benzoxazin-2-one;
(2E)-3-(4,4-Dimethyl-2-oxo-1,4-dihydro-2H-3,1-benzoxazin-6-yl)acrylamide;
(2E)-3-(4,4-Dimethyl-2-oxo-1,4-dihydro-2H-3,1-benzoxazin-6-yl)but-2-enamide;
(2E)-3-(4,4-Dimethyl-2-oxo-1,4-dihydro-2H-3,1-benzoxazin-6-yl)-3-phenylacrylonitrile;
(2E)-3-(4,4-Dimethyl-2-oxo-1,4-dihydro-2H-3,1-benzoxazin-6-yl)-3-phenylacrylamide;
(2E)-3-(4,4-Dimethyl-2-oxo-1,4-dihydro-2H-3,1-benzoxazin-6-yl)acrylonitrile;
Methyl(2E)-3-(4,4-dimethyl-2-oxo-1,4-dihydro-2H-3,1-benzoxazin-6-yl)but-2-enoate;
Methyl(2E)-3-(4,4-dimethyl-2-oxo-1,4-dihydro-2H-3,1-benzoxazin-6-yl)-3-phenylacrylate;
(2E)-3-(4,4-Dimethyl-2-oxo-1,4-dihydro-2H-3,1-benzoxazin-6-yl)but-2-enenitrile;
(2E)-3-(4,4-Dimethyl-2-oxo-1,4-dihydro-2H-3,1-benzoxazin-6-yl)acrylic acid;
(2E)-3-(4,4-Dimethyl-2-oxo-1,4-dihydro-2H-3,1-benzoxazin-6-yl)but-2-enoic acid;
(2E)-3-(2-Oxo-1,2-dihydrospiro[3,1-benzoxazine-4,1′-cyclohexan]-6-yl)but-2-enamide;
(2E)-3-(2-oxo-1,2-dihydrospiro[3,1-benzoxazine-4,1′-cyclohexan]-6-yl)but-2-enenitrile;
(2E)-3-(2-Oxo-1,2-dihydrospiro[3,1-benzoxazine-4,1′-cyclohexan]-6-yl)pent-2-enamide;
(2E)-3-(2-Oxo-1,2-dihydrospiro[3,1-benzoxazine-4,1′-cyclohexan]-6-yl)pent-2-enenitrile;
(2E)-3-(2-Oxo-1,2-dihydrospiro[3,1-benzoxazine-4,1′-cyclohexan]-6-yl)hex-2-enenitrile;
(2E)-4-methyl-3-(2-oxo-1,2-dihydrospiro[3,1-benzoxazine-4,1′-cyclohexan]-6-yl)pent-2-enenitrile;
(2E)-3-(4,4-Diethyl-2-oxo-1,4-dihydro-2H-3,1-benzoxazin-6-yl)but-2-enenitrile;
(2E)-3-(4,4-Diethyl-2-oxo-1,4-dihydro-2H-3,1-benzoxazin-6-yl)pent-2-enenitrile;
(2E)-3-(4,4-Diethyl-2-oxo-1,4-dihydro-2H-3,1-benzoxazin-6-yl)hex-2-enamide;
(2E)-3-(4,4-Diethyl-2-oxo-1,4-dihydro-2H-3,1-benzoxazin-6-yl)hex-2-enenitrile;
(2E)-3-(4,4-Diethyl-2-oxo-1,4-dihydro-2H-3,1-benzoxazin-6-yl)hex-2-enal;
(2E)-3-(4,4-Diethyl-2-oxo-1,4-dihydro-2H-3,1-benzoxazin-6-yl)-4-methylpent-2-enenitrile;
(2E)-3-(4,4-Dimethyl-2-oxo-1,4-dihydro-2H-3,1-benzoxazin-6-yl)pent-2-enenitrile;
(2E)-3-(4,4-Dimethyl-2-oxo-1,4-dihydro-2H-3,1-benzoxazin-6-yl)hex-2-enenitrile; and
(2E)-3-(4,4-Dimethyl-2-oxo-1,4-dihydro-2H-3,1-benzoxazin-6-yl)-4-methylpent-2-enenitrile, and
pharmaceutically acceptable salts thereof.

9. The compound according to claim 6, selected from the group consisting of:
Methyl(2Z)-3-(4,4-dimethyl-2-oxo-1,2,3,4-tetrahydroquinolin-6-yl)acrylate;
Methyl(2Z)-3-(4,4-dimethyl-2-oxo-1,4-dihydro-2H-3,1-benzoxazin-6-yl)acrylate;
6-[(Z)-2-Isoxazol-5-ylvinyl]-4,4-dimethyl-1,4-dihydro-2H-3,1-benzoxazin-2-one;
(2Z)-3-(4,4-Dimethyl-2-oxo-1,4-dihydro-2H-3,1-benzoxazin-6-yl)acrylamide;
(2Z)-3-(4,4-Dimethyl-2-oxo-1,4-dihydro-2H-3,1-benzoxazin-6-yl)but-2-enamide;
(2Z)-3-(4,4-Dimethyl-2-oxo-1,4-dihydro-2H-3,1-benzoxazin-6-yl)-3-phenylacrylonitrile;
(2Z)-3-(4,4-Dimethyl-2-oxo-1,4-dihydro-2H-3,1-benzoxazin-6-yl)-3-phenylacrylamide;
(2Z)-3-(4,4-Dimethyl-2-oxo-1,4-dihydro-2H-3,1-benzoxazin-6-yl)acrylonitrile;
Methyl(2Z)-3-(4,4-dimethyl-2-oxo-1,4-dihydro-2H-3,1-benzoxazin-6-yl)but-2-enoate;
Methyl(2Z)-3-(4,4-dimethyl-2-oxo-1,4-dihydro-2H-3,1-benzoxazin-6-yl)-3-phenylacrylate;
(2Z)-3-(4,4-Dimethyl-2-oxo-1,4-dihydro-2H-3,1-benzoxazin-6-yl)but-2-enenitrile;
(2Z)-3-(4,4-Dimethyl-2-oxo-1,4-dihydro-2H-3,1-benzoxazin-6-yl)acrylic acid;
(2Z)-3-(4,4-Dimethyl-2-oxo-1,4-dihydro-2H-3,1-benzoxazin-6-yl)but-2-enoic acid;
(2Z)-3-(2-Oxo-1,2-dihydrospiro[3,1-benzoxazine-4,1′-cyclohexan]-6-yl)but-2-enamide;
(2Z)-3-(2-oxo-1,2-dihydrospiro[3,1-benzoxazine-4,1′-cyclohexan]-6-yl)but-2-enenitrile;
(2Z)-3-(2-Oxo-1,2-dihydrospiro[3,1-benzoxazine-4,1′-cyclohexan]-6-yl)pent-2-enamide;
(2Z)-3-(2-Oxo-1,2-dihydrospiro[3,1-benzoxazine-4,1′-cyclohexan]-6-yl)pent-2-enenitrile;
(2Z)-3-(2-Oxo-1,2-dihydrospiro[3,1-benzoxazine-4,1′-cyclohexan]-6-yl)hex-2-enenitrile;
(2Z)-4-methyl-3-(2-oxo-1,2-dihydrospiro[3,1-benzoxazine-4,1′-cyclohexan]-6-yl)pent-2-enenitrile;
(2Z)-3-(4,4-Diethyl-2-oxo-1,4-dihydro-2H-3,1-benzoxazin-6-yl)but-2-enenitrile;
(2Z)-3-(4,4-Diethyl-2-oxo-1,4-dihydro-2H-3,1-benzoxazin-6-yl)pent-2-enenitrile;
(2Z)-3-(4,4-Diethyl-2-oxo-1,4-dihydro-2H-3,1-benzoxazin-6-yl)hex-2-enamide;
(2Z)-3-(4,4-Diethyl-2-oxo-1,4-dihydro-2H-3,1-benzoxazin-6-yl)hex-2-enenitrile;
(2Z)-3-(4,4-Diethyl-2-oxo-1,4-dihydro-2H-3,1-benzoxazin-6-yl)hex-2-enal;
(2Z)-3-(4,4-Diethyl-2-oxo-1,4-dihydro-2H-3,1-benzoxazin-6-yl)-4-methylpent-2-enenitrile;
(2Z)-3-(4,4-Dimethyl-2-oxo-1,4-dihydro-2H-3,1-benzoxazin-6-yl)pent-2-enenitrile;
(2Z)-3-(4,4-Dimethyl-2-oxo-1,4-dihydro-2H-3,1-benzoxazin-6-yl)hex-2-enenitrile; and
(2Z)-3-(4,4-Dimethyl-2-oxo-1,4-dihydro-2H-3,1-benzoxazin-6-y)-4-methylpent-2-enenitrile, and
pharmaceutically acceptable salts thereof.

10. A method for preparing a compound according to claim 1, said method comprising reacting a compound of formula (II) with a vinyl compound of formula (III): 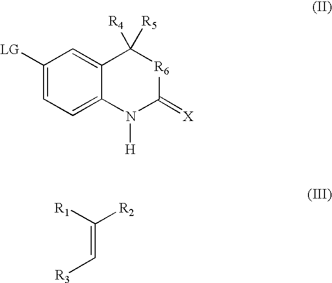
wherein LG is a leaving group.

11. The method according to claim 10, which is performed in the presence of a catalyst and phosphine.

12. A method for preparing a compound according to claim 1, wherein R₁ or R₂ is —CN, said method comprising reacting an amide of formula (IVa) or (IVb) with a dehydrating agent

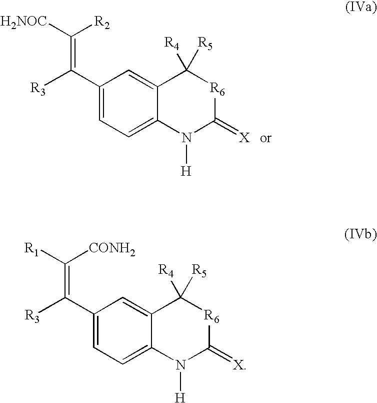

13. The method according to claim 12, wherein said dehydrating agent is thionyl chloride, phosphorus oxychloride, phosphorus pentachloride, phosgene, oxalyl chloride, methanesulfonyl chloride or acetic anhydride.

14. The method according to claim 12, which is performed at elevated temperatures.

15. The method according to claim 12, which is performed at temperatures of about room temperature to the reflux temperature of said solvent.

16. A pharmaceutical composition comprising a pharmaceutically acceptable carrier and a compound of claim 1.

17. A method of treating endometriosis, hormone-dependent carcinomas, or leiomyoma, fibroids, dysfunctional bleeding, polycystic ovary syndrome, menopause related symptoms, symptoms of premenstrual syndrome, or symptoms of premenstrual dysphoric disorder, comprising delivering to a subject in need thereof a compound of claim 1.

18. The method according to claim 17, wherein said fibroids are uterine fibroids.

19. A method of contraception or hormone replacement therapy in a female comprising delivering a compound of claim 1 to a female subject.

20. A method of stimulating food intake or synchronizing estrus comprising administering to a mammal in need thereof a compound of claim 1.

21. A kit comprising a compound of claim 1 and a carrier suitable for administration to a mammalian subject.

22. A compound of formula (Ia), or a pharmaceutically acceptable salt thereof: 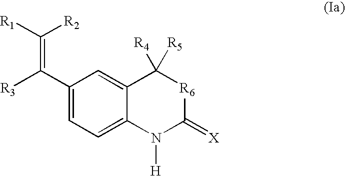
wherein:
R₁ is selected from the group consisting of H, —CO₂—C₁—C₃ alkyl, —CO₂H, —CR₇O, —CONR₈R₉, —CN, and isoxazole;
R₂ is selected from the group consisting of H, —CO₂—C₁—C₃-alkyl, —CO₂H, —CR₇O, —CONR₈R₉, —CN, C₁-C₆ alkyl, aryl, and heteroaryl;
with the proviso that both R₁ and R₂ are not H;
R₃ is H, C₁-C₆ alkyl, aryl, or heteroaryl;
R4 and R₅ are independently C₁-C₃ alkyl or together with the carbon to which they are attached form a saturated 3 to 6-membered carbon-based ring;
R₆ is O or CR₁₀R₁₁;
R₇, R₈ and R₉ are independently selected from the group consisting of H and C₁-C₃ alkyl;
R₁₀ and R₁₁ are independently H or C₁-C₃ alkyl; or
R₁₀ and R₁₁ together with the carbon to which they are attached form a saturated 3 to 6-membered carbon-based ring; and
X is O or S.

23. A compound of formula (Ib), or a pharmaceutically acceptable salt thereof: 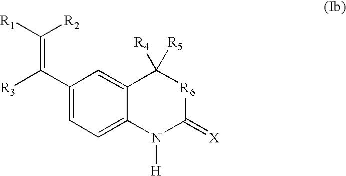
wherein:
R₁ and R₃ together form a 6-membered lactone, wherein a 2-carbon bridge is formed between the CO₂—C₁—C₃ alkyl of R₁ and an alkyl group of R₃;
R₂ is selected from the group consisting of H, —CO₂—C₁—C₃-alkyl, —CO₂H, —CR₇O, —CONR₈R₉, —CN, C₁-C₆ alkyl, aryl, and heteroaryl;
R₄ and R₅ are independently C₁-C₃ alkyl or together with the carbon to which they are attached form a saturated 3 to 6-membered carbon-based ring;
R₆ is O or CR₁₀R₁₁;
R₇, R₈ and R₉ are independently selected from among H and C₁-C₃ alkyl;
R₁₀ and R₁₁ are independently H or C₁-C₃ alkyl; or
R₁₀ and R₁₁ together with the carbon to which they are attached form a saturated 3 to 6-membered carbon-based ring; and
X is O or S.
